(19) United States
(12) Patent Application Publication
(10) Pub. No.: US 2025/0249010 A1
(43) Pub. Date: Aug. 7, 2025

(54) METHODS FOR TREATING PRIMARY MEMBRANOUS NEPHROPATHY

(71) Applicant: BEIGENE SWITZERLAND GMBH, Basel (CH)

(72) Inventors: Zhen Yao, Beijing (CN); Yanyan Chen, Shanghai (CN)

(21) Appl. No.: 19/188,946

(22) Filed: Apr. 24, 2025

Related U.S. Application Data
(63) Continuation of application No. PCT/CN2023/126564, filed on Oct. 25, 2023.

(30) Foreign Application Priority Data
Oct. 26, 2022 (WO) PCT/CN2022/127565
Jan. 11, 2023 (WO) PCT/CN2023/071765

(51) Int. Cl. A61K 31/519

(52) U.S. Cl. CPC A61K 31/519

(57) ABSTRACT

Provided here are methods of treating primary membranous nephropathy in a patient in need thereof, comprising administering to said patient a BTK inhibitor, e.g., (S)-7-(1-acryloylpiperidin-4-yl)-2-(4-phenoxyphenyl)-4,5,6,7-tetrahydropyrazolo[1,5-a]pyrimidine-3-carboxamide, or a pharmaceutically acceptable salt, tautomer, stereoisomer, enantiomer, isotopologue, solvate, or prodrug thereof.

### FIELD

Provided herein are methods and materials involved in diagnosing and/or treating mammals having primary membranous nephropathy (e.g., primary membranous nephropathy with an elevated level of PLA2R and/or THSD7A in the glomerular basement membrane (GBM)). For example, provided herein are methods and materials for administering a Bruton's tyrosine kinase (BTK) inhibitor to treat a mammal (e.g., a human) having primary membranous nephropathy.

### BACKGROUND

Membranous nephropathy is one of the most common causes of nephrotic syndrome. It is classified into primary and secondary membranous nephropathy. Primary membranous nephropathy accounts for 70-75% cases and secondary for 25-30% cases. Membranous nephropathy results from an autoimmune response to accumulation of a target antigen in the GBM. The GBM is the integral part of the glomerular capillaries forming the filtering unit of the kidney. It is reported that in about 70 percent of primary cases, membranous nephropathy results from accumulation of phospholipase A2 receptor (PLA2R) target antigens in the GBM and subsequent formation of antigen-antibody complexes (Beck et al., N. Engl. J. Med., 361:11-21 (2009)). Thrombospondin type-1 domain-containing 7A (THSD7A) is reported to be another antigen that accounts for about 5% of the cases of primary membranous nephropathy (Tomas et al., N. Engl. J. Med., 24:2277-2287 (2014)). Secondary membranous nephropathy is often associated with autoimmune diseases such as lupus, mixed connective tissue disorder, and Sjogren's syndrome. The target antigens in the remaining 25% primary membranous nephropathy are under research.

Though there have been many recent advances in the treatment of primary membranous nephropathy, there remains a need for more effective and/or enhanced treatments.

Citation or identification of any reference in this section is not to be construed as an admission that the reference is prior art to the present application.

### SUMMARY

Provided herein is a method of treating a patient having primary membranous nephropathy, wherein the method comprises administering to the patient a Bruton's Tyrosine Kinase inhibitor. In some embodiments, the Bruton's Tyrosine Kinase inhibitor is Compound A having the name of (S)-7-(1-acryloylpiperidin-4-yl)-2-(4-phenoxyphenyl)-4,5,6,7-tetrahydropyrazolo[1,5-a]pyrimidine-3-carboxamide or the following structure:

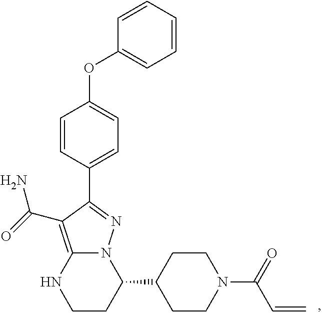 [SMILES: C=CC(=O)N1CCC([C@@H]2CCNc3c(C(N)=O)c(-c4ccc(Oc5ccccc5)cc4)nn32)CC1]

or a pharmaceutically acceptable salt, tautomer, stereoisomer, enantiomer, isotopologue, solvate, or prodrug thereof.

In some embodiments, the patient is identified as having (i) an elevated level of an autoantibody specific for an antigen or (ii) kidney tissue comprising an elevated level of the antigen. In one embodiment, the antigen is expressed by the human podocyte, such as PLA2R, or THSD7A. In some embodiments, the antigen is PLA2R. In some embodiments, the level of an autoantibody specific for PLA2R is more than about 20 RU/mL, about 40 RU/mL, about 50 RU/mL, about 60 RU/mL, or about 80 RU/mL in sera prior to the administering step. In some embodiments, the antigen is THSD7A. In some embodiments, the level of autoantibodies present within the patient is reduced by at least about 5 percent, about 25 percent, or about 50 percent following the administering step.

In some embodiments, Compound A or a pharmaceutically acceptable salt, tautomer, stereoisomer, enantiomer, isotopologue, solvate, or prodrug thereof is administered one, two, or three times a day. In some embodiments, Compound A or a pharmaceutically acceptable salt, tautomer, stereoisomer, enantiomer, isotopologue, solvate, or prodrug thereof is administered twice a day. In some embodiments, Compound A or a pharmaceutically acceptable salt, tautomer, stereoisomer, enantiomer, isotopologue, solvate, or prodrug thereof is administered once a day.

In some embodiments, Compound A or a pharmaceutically acceptable salt, tautomer, stereoisomer, enantiomer, isotopologue, solvate, or prodrug thereof is administered from about 40 mg to about 320 mg per day. In some embodiments, Compound A or a pharmaceutically acceptable salt, tautomer, stereoisomer, enantiomer, isotopologue, solvate, or prodrug thereof is administered at about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, or about 320 mg per day. In some embodiments, Compound A or a pharmaceutically acceptable salt, tautomer, stereoisomer, enantiomer, isotopologue, solvate, or prodrug thereof is administered at about 40 mg, about 80 mg, about 160 mg, or about 320 mg per day. In one embodiment, the method provides a plasma Compound A AUC₀₋₂₄ₕ between about 1,607 ng*h/ml and about 2,984 ng*h/ml in the patient. In one embodiment, the method provides a plasma Compound A AUC₀₋₂₄ₕ between about 1,836 ng*h/ml and about 2,754 ng*h/ml in the patient. In one embodiment, the method provides a plasma Compound A AUC₀₋₂₄ₕ between about 2,066 ng*h/ml and about 2,525 ng*h/ml in the patient. In one embodiment, the method provides a plasma Compound A AUC₀₋₂₄ₕ about 2295 ng*h/ml in the patient. In one embodiment, the method provides a plasma Compound A AUC₀₋₂₄ₕ about 2180 ng*h/ml in the patient.

In some embodiments, the patient achieves a partial remission, or a complete remission.

### BRIEF DESCRIPTION OF DRAWINGS

FIG. 1 depicts the study schema.

FIG. 2 depicts the multiple testing strategy for Part 2.

### DETAILED DESCRIPTION

### Definitions

As used herein, “Compound A” refers to the compound having the name of (S)-7-(1-acryloylpiperidin-4-yl)-2-(4-phenoxyphenyl)-4,5,6,7-tetrahydropyrazolo[1,5-a]pyrimidine-3-carboxamide, or the structure of formula (I):
(I),
   or a pharmaceutically acceptable salt, tautomer, stereoisomer, enantiomer, isotopologue, solvate, or prodrug thereof. Compound A is disclosed and claimed, along with pharmaceutically acceptable salts thereof, and also as solvates thereof, as being useful as an inhibitor of BTK activity, in WO2014/173289 and WO2018/033853, the entire disclosure of which are incorporated herein by reference. Compound A is compound 27b in WO2014/173289 and Compound 1 in WO2018/033853. Compound A can be prepared as described in WO2014/173289 and WO2018/033853. Unless specifically stated, “Compound A” as used herein refers to Compound A or a pharmaceutically acceptable salt, tautomer, stereoisomer, enantiomer, isotopologue, solvate, or prodrug thereof. Compound A disclosed herein may contain one or more chiral atoms, or may otherwise be capable of existing as enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A. As used herein, all amounts specified for Compound A are indicated as the amount of free or unsalted compound.

In one embodiment, a solid form of Compound A is used for the treatment provided herein. In one embodiment, a crystal form of Compound A is used for the treatment provided herein. In one embodiment, an amorphous form of Compound A is used for the treatment provided herein. In one embodiment, the freebase of Compound A is used for the treatment provided herein. In one embodiment, Form A of Compound 1 of WO201833853 is used for the treatment provided herein. In some embodiments, Form A has an X-ray powder diffraction pattern comprising diffraction peaks having 2θ angle values independently selected from: approximately 14.8±0.2°, 16.4±0.2° and 21.4±0.2°. In some embodiments, Form A has an X-ray powder diffraction pattern comprising diffraction peaks having 2θ angle values independently selected from: approximately 14.8±0.2°, 15.6±0.2°, 16.4±0.2° and 21.4±0.2°. In some embodiments, Form A has an X-ray powder diffraction pattern comprising diffraction peaks having 2θ angle values independently selected from: approximately 12.2±0.2°, 12.9±0.2°, 14.8±0.2°, 15.6±0.2°, 16.4±0.2° and 21.4±0.2°. In some embodiments, Form A has an X-ray powder diffraction pattern comprising diffraction peaks having 2θ angle values independently selected from: approximately 12.2±0.2°, 12.9±0.2°, 14.8±0.2°, 15.6±0.2°, 16.4±0.2°, 17.7±0.2°, 18.5±0.2°, 20.7±0.2° and 21.4±0.2°.

The term “BTK inhibitor,” as used herein, refers to compounds having inhibitory activity against Bruton's Tyrosine Kinase (BTK). Exemplary BTK inhibitors include those described above: ibrutinib, acalabrutinib, evobrutinib, fenebrutinib, poseltinib, vecabrutinib, tirabrutinib, spebrutinib, and zanubrutinib.

The term “autoantibody,” as used herein, refers to its general meaning in the art and refers to an antibody that is produced by the immune system of a subject (or patient) and that is directed against subject's (or patient's) own antigens (for example PLA2R or THSD7A). Immune complexes which comprise autoantibodies and their specific antigens may attack the body's own cells, tissues, and/or organs, causing inflammation and cell injury. The terms “autoantibody recognizing PLA2R” and “autoantibody specific to PLA2R” can be used interchangeably with “anti-PLA2R autoantibody”. The terms “autoantibody recognizing THSD7A” and “autoantibody specific to THSD7A” can be used interchangeably with “anti-THSD7A autoantibody”.

The term “PLA2R,” as used herein, refers to its general meaning in the art (secretory phospholipase A2 receptor, also known as PLA2R1) and herein refers to the M-type phospholipase A2 receptor, a receptor encoded in humans by the PLA2R1 gene, particularly known as a major antigen in idiopathic membranous nephropathy, An exemplary human native PLA2R1 amino acid sequence is provided in NP_001007268 (GenPept database) and an exemplary human native PLA2R1 mRNA sequence is provided in NM_001007267 (GenBank database). It is noteworthy that all reference to database such as codes or accession numbers disclosed herein refer to the versions available online on Oct. 10, 2022.

The term “THSD7A,” as used herein, refers to its general meaning in the art (e.g., Thrombospondin Type I Domain Containing 7A) and refers to a protein of 1657 amino acids highly expressed on podocytes and involved in endothelial cell migration and filopodia formation during angiogenesis via a FAK-dependent mechanism. The term may include naturally occurring THSD7A and variants and modified forms thereof. THSD7A may be from any source, but typically is a mammalian (e.g., human and non-human primate or other mammalian species) THSD7A, particularly a human THSD7A. An exemplary human native THSD7A amino acid sequence is provided in Q9UPZ6 (UniProt database) and NP_056019 (GenPept database, precursor sequence) and an exemplary human native THSD7A mRNA sequence is provided in NM_015204 (GenBank database, precursor sequence). It is noteworthy that all reference to database such as codes or accession numbers disclosed herein refer to the versions available online on Oct. 10, 2022.

The term “antigen” or “Ag” as used herein is defined as a molecule that can provoke an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or can be macromolecules besides a polypeptide. Such a biological sample can include, but is not limited to a tissue sample, a tumor sample, a cell or fluid with other biological components.

By the term “treating” and derivatives thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition (3) to alleviate one or more of the symptoms, effects or side effects associated with the condition or one or more of the symptoms, effects or side effects associated with the condition or treatment thereof, or (4) to slow the progression of the condition or one or more of the biological manifestations of the condition.

As used herein, the term “effective amount” means that amount of a drug or pharmaceutical agent that elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “solvate” refers to a complex of variable stoichiometry formed by a solute (in this invention, compounds of formula (I) or a salt thereof and a solvent. Also, it is understood that Compound A may be presented, separately or both, as solvates. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent. Examples of suitable pharmaceutically acceptable solvents include, without limitation, water, ethanol and acetic acid. In another embodiment, the solvent used is water (i.e., a hydrate).

Compound A may have the ability to crystallize in more than one form, a characteristic, which is known polymorphism, and it is understood that such polymorphic forms (“polymorphs”) are within the scope of Compound A. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percentages of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degrees 2θ) while still describing the particular XRPD peak.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds provided herein include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center is substantially free of the opposite enantiomer of the compound. A stereomerically pure compound having two chiral centers is substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

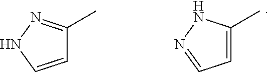

As readily understood by one skilled in the art, a wide variety of functional groups and other structures may exhibit tautomerism and all tautomers of the compounds provided herein are within the scope of the present invention.

The term “subject” includes an animal, including, but not limited to, an animal such a cow, monkey, horse, sheep, pig, chicken, turkey, quail, cat, dog, mouse, rat, rabbit, or guinea pig, in one embodiment a mammal, in another embodiment a human.

While it is possible that, for use in therapy, Compound A, may be administered as the raw chemical, it is possible to present the active ingredient as a pharmaceutical composition. Accordingly, the invention further provides pharmaceutical compositions, which include a Compound A, and one or more pharmaceutically acceptable carriers, diluents, or excipients. Compound A is as described above. The carrier(s), diluent(s) or excipient(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical composition including admixing a Compound A with one or more pharmaceutically acceptable carriers, diluents, or excipients.

Pharmaceutical compositions may be presented in unit dose forms containing a predetermined amount of active ingredient per unit dose. As is known to those skilled in the art, the amount of active ingredient per dose depends on the condition being treated, the route of administration and the age, weight, and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

Compound A may be administered by any appropriate route. Suitable routes include oral, rectal, nasal, topical (including buccal and sublingual), vaginal, and parenteral (including subcutaneous, intramuscular, intravenous, intradermal, intrathecal, and epidural).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

By the term “kit” “or kit of parts” as used herein is meant the pharmaceutical composition or compositions that are used to administer Compound A according to the disclosure. In one embodiment, the kit can contain Compound A in a single pharmaceutical composition, such as a tablet, or in separate pharmaceutical compositions. In one aspect, there is provided a kit of parts comprising components: Compound A in association with a pharmaceutically acceptable excipients, diluents, or carrier. The kit can also be provided with instruction, such as dosage and administration instructions. Such dosage and administration instructions can be of the kind that are provided to a doctor, for example by a drug product label, or they can be of the kind that are provided by a doctor, such as instructions to a patient.

The term “dose” as used herein is understood to mean a dose that is intended to either slowly raise plasma or blood concentration levels of the compound to a therapeutically effective level, or to maintain such a therapeutically effective level.

The term “elevated level” as used herein with respect to a polypeptide, antigen, or antibody level refers to a level of the polypeptide, antigen, or antibody that is greater (e.g., at least 10, 25, 35, 45, 50, 55, 65, 75, 80, 90, or 100 percent greater) than the median level of the polypeptide, antigen, or antibody present within normal tissue or sera of comparable mammals not having primary membranous nephropathy.

### Kits

Provided herein is a kit comprising a compound provided herein and means for monitoring patient response to administration of said compound provided herein.

In other embodiments, provided herein are kits comprising a compound provided herein and means for measuring the amount of inhibition of BTK in a patient. In certain embodiments, the kits comprise means for measuring inhibition of BTK in circulating plasma, serum, blood, or tumor cells and/or skin biopsies or kidney biopsies/aspirates of a patient. In certain embodiments, provided herein are kits comprising a compound provided herein and means for measuring the amount of inhibition of BTK before, during and/or after administration of a compound provided herein.

In certain embodiments, the kits provided herein further comprise instructions for use, such as for administering a compound provided herein and/or monitoring patient response to administration of the compound.

### Method of Treatment

Provided herein is a method of treating a patient having primary membranous nephropathy, wherein the method comprises administering to the patient a Bruton's Tyrosine Kinase inhibitor. Provided herein is a Bruton's Tyrosine Kinase inhibitor for use in treating or preventing primary membranous nephropathy. Provided here is use of a Bruton's Tyrosine Kinase inhibitor in the manufacture of medicament for treating or preventing primary membranous nephropathy.

In some embodiments, the patient is a human.

In some embodiments, the Bruton's Tyrosine Kinase inhibitor is Compound A having the name of (S)-7-(1-acryloylpiperidin-4-yl)-2-(4-phenoxyphenyl)-4,5,6,7-tetrahydropyrazolo[1,5-a]pyrimidine-3-carboxamide or the following structure:

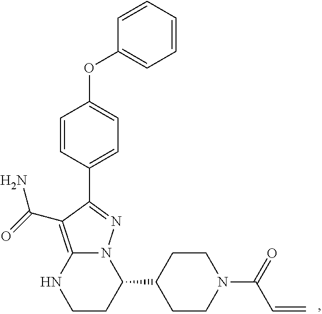 [SMILES: C=CC(=O)N1CCC([C@@H]2CCNc3c(C(N)=O)c(-c4ccc(Oc5ccccc5)cc4)nn32)CC1]

or a pharmaceutically acceptable salt, tautomer, stereoisomer, enantiomer, isotopologue, solvate, or prodrug thereof.

In some embodiments, the patient is identified as having (i) an elevated level of an autoantibody specific for an antigen, or (ii) kidney tissue comprising an elevated level of the antigen. In one embodiment, the antigen is expressed by the human podocyte. In one embodiment, the antigen is expressed by the human podocyte, such as PLA2R, or THSD7A. In some embodiments, the antigen is PLA2R, or THSD7A. In some embodiments, the antigen is PLA2R. In some embodiments, the antigen is THSD7A. In one embodiment, the patient is identified as biopsy confirmed primary membranous nephropathy and with persistent nephrotic syndrome after run-in period. In one embodiment, the patient is a PLA2R mediated patient with primary membranous nephropathy. In one embodiment, the patient is a THSD7A mediated patient with primary membranous nephropathy.

In some embodiments, the patient is identified as having an elevated level of the autoantibody. In some embodiments, the level of autoantibodies present within the patient is reduced by at least about 5 percent, at least about 25 percent, or at least about 50 percent following the administering step. In some embodiments, the level of autoantibodies present in sera within the patient is reduced by at least about 5 percent, at least about 25 percent, or at least about 50 percent following the administering step. In some embodiments, the level of autoantibodies present within the subject (e.g., in sera) is reduced by at least about 5 percent following the administering step. In some embodiments, the level of autoantibodies present within the subject (e.g., in sera) is reduced by at least about 25 percent following the administering step. In some embodiments, the level of autoantibodies present within the subject (e.g., in sera) is reduced by at least about 50 percent following the administering step.

In some embodiments, the patient is identified as having an elevated level of the autoantibody specific to an antigen in sera. In some embodiments, the antigen is PLA2R. In some embodiments, the antigen is THSD7A. In some embodiments, the level of an autoantibody specific for PLA2R is more than about 20 RU/mL, about 40 RU/mL, about 50 RU/mL, about 60 RU/mL, or about 80 RU/mL in sera prior to the administering step. In some embodiments, the level of an autoantibody specific for PLA2R is more than about 20 RU/mL in sera prior to the administering step. In some embodiments, the level of an autoantibody specific for PLA2R is more than about 40 RU/mL in sera prior to the administering step. In some embodiments, the level of an autoantibody specific for PLA2R is more than about 50 RU/mL in sera prior to the administering step. In some embodiments, the level of an autoantibody specific for PLA2R is more than about 60 RU/mL in sera prior to the administering step. In some embodiments, the level of an autoantibody specific for PLA2R is more than about 80 RU/mL in sera prior to the administering step.

In some embodiments, the patient is identified as having the kidney tissue comprising an elevated level of an antigen. In some embodiments, the level of the antigen present within kidney tissue of the patient is reduced by at least about 5 percent, at least about 25 percent, or at least about 50 percent following the administering step. In some embodiments, the level of the antigen present within kidney tissue of the patient is reduced by at least about 5 percent following the administering step. In some embodiments, the level of the antigen present within kidney tissue of the patient is reduced by at least about 25 percent following the administering step. In some embodiments, the level of the antigen present within kidney tissue of the patient is reduced by at least about 50 percent following the administering step.

In some embodiments, Compound A or a pharmaceutically acceptable salt, tautomer, stereoisomer, enantiomer, isotopologue, solvate, or prodrug thereof is administered one to three times a day. In some embodiments, Compound A or a pharmaceutically acceptable salt, tautomer, stereoisomer, enantiomer, isotopologue, solvate, or prodrug thereof is administered once a day. In some embodiments, Compound A or a pharmaceutically acceptable salt, tautomer, stereoisomer, enantiomer, isotopologue, solvate, or prodrug thereof is administered twice a day. In some embodiments, Compound A or a pharmaceutically acceptable salt, tautomer, stereoisomer, enantiomer, isotopologue, solvate, or prodrug thereof is administered three times a day.

In some embodiments, Compound A or a pharmaceutically acceptable salt, tautomer, stereoisomer, enantiomer, isotopologue, solvate, or prodrug thereof is administered from about 40 mg to about 320 mg per day.

In some embodiments, Compound A or a pharmaceutically acceptable salt, tautomer, stereoisomer, enantiomer, isotopologue, solvate, or prodrug thereof is administered at about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, or about 320 mg per day.

In some embodiments, Compound A or a pharmaceutically acceptable salt, tautomer, stereoisomer, enantiomer, isotopologue, solvate, or prodrug thereof is administered at about 40 mg, about 80 mg, about 160 mg, or about 320 mg per day. In some embodiments, Compound A or a pharmaceutically acceptable salt, tautomer, stereoisomer, enantiomer, isotopologue, solvate, or prodrug thereof is administered at about 160 mg, or about 320 mg per day.

In some embodiments, the method comprises administering to the patient Compound A at a dose of about 160 mg twice a day (BID) or about 160 mg once a day (QD).

In some embodiments, the method comprises administering to the patient Compound A at a dose of about 160 mg twice a day (BID) or about 160 mg once a day (QD) orally.

In some embodiments, the method comprises administering to the patient Compound A at a dose of about 160 mg twice a day (BID) orally.

In some embodiments, the method comprises administering to the patient Compound A at a dose of about 160 mg once a day (QD) orally.

In some embodiments, the patient achieves a partial remission, or a complete remission.

In some embodiments, the patient achieves a partial remission.

In some embodiments, the patient achieves a complete remission.

In some cases, a course of treatment and/or the severity of one or more symptoms related to membranous nephropathy can be monitored. Any appropriate method can be used to determine whether or not membranous nephropathy is being treated. For example, immunological techniques (e.g., ELISA) can be performed to determine if the level of autoantibodies (e.g., anti-PLA2R autoantibodies, or anti-THSD7A autoantibodies) present within a mammal being treated as described herein is reduced following the administration of one or more BTK inhibitors. Remission and relapse of the disease can be monitored by testing for one or more markers for membranous nephropathy. In some cases, remission can be ascertained by detecting the disappearance or reduction of autoantibodies specific to THSD7A, PLA2R, in the sera. In some cases, relapse of membranous nephropathy can be ascertained by a reappearance or elevation of autoantibodies to THSD7A, PLA2R, in the sera.

In some cases, remission may be predicted by detecting the disappearance or reduction of autoantibodies specific to THSD7A, or PLA2R, in the sera. In some cases, relapse of membranous nephropathy may be predicted by a reappearance or elevation of autoantibodies to THSD7A, or PLA2R, in the sera.

In one embodiment, Compound A is administered one to three times a day. In one embodiment, Compound A is administered three times a day. In one embodiment, Compound A is administered twice a day. In one embodiment, Compound A is administered once a day.

In one embodiment, the method provides a plasma Compound A AUC₀₋₂₄ₕ between about 1,607 ng*h/ml and about 2,984 ng*h/ml in the patient. In one embodiment, the method provides a plasma Compound A AUC₀₋₂₄ₕ between about 1,836 ng*h/ml and about 2,754 ng*h/ml in the patient. In one embodiment, the method provides a plasma Compound A AUC₀₋₂₄ₕ between about 2,066 ng*h/ml and about 2,525 ng*h/ml in the patient. In one embodiment, the method provides a plasma Compound A AUC₀₋₂₄ₕ about 2295 ng*h/ml in the patient. In one embodiment, the method provides a plasma Compound A AUC₀₋₂₄ₕ about 2180 ng*h/ml in the patient.

In one embodiment, the method provides a plasma Compound A AUC_{0-∞} between about 1,622 ng*h/ml and about 3,786 ng*h/ml in the patient. In one embodiment, the method provides a plasma Compound A AUC_{0-∞} between about 2,163 ng*h/ml and about 3,245 ng*h/ml in the patient. In one embodiment, the method provides a plasma Compound A AUC_{0-∞} between about 2,434 ng*h/ml and about 2,974 ng*h/ml in the patient. In one embodiment, the method provides a plasma Compound A AUC_{0-∞} about 2704 ng*h/ml in the patient.

In one embodiment, the method provides a plasma Compound A AUC_{0-∞} between about 1,087 ng*h/ml and about 2,535 ng*h/ml in the patient. In one embodiment, the method provides a plasma Compound A AUC_{0-∞} between about 1,449 ng*h/ml and about 2,173 ng*h/ml in the patient. In one embodiment, the method provides a plasma Compound A AUC_{0-∞} between about 1,630 ng*h/ml and about 1,992 ng*h/ml in the patient. In one embodiment, the method provides a plasma Compound A AUC_{0-∞} about 1,811 ng*h/ml in the patient.

In one embodiment, the method provides a plasma Compound A AUC_{0-∞} between about 843 ng*h/ml and about 1,967 ng*h/ml in the patient. In one embodiment, the method provides a plasma Compound A AUC_{0-∞} between about 1,124 ng*h/ml and about 1,686 ng*h/ml in the patient. In one embodiment, the method provides a plasma Compound A AUC_{0-∞} between about 1,265 ng*h/ml and about 1,546 ng*h/ml in the patient. In one embodiment, the method provides a plasma Compound A AUC_{0-∞} about 1,405 ng*h/ml in the patient.

In one embodiment, the method provides a plasma Compound A AUC_{0-∞} between about 843 ng*h/ml and about 3,786 ng*h/ml in the patient.

In some embodiments, the AUC₀₋₂₄ₕ and AUC_{0-∞} are measured in the subject's plasma. In some embodiments, the AUC₀₋₂₄ₕ and AUC_{0-∞} are measured in the subject's blood.

In one embodiment, the subject achieves a partial remission, or a complete remission. In one embodiment, the subject achieves a partial remission. In one embodiment, the subject achieves a complete remission. In one embodiment, the subject achieves a partial remission, or a complete remission for 1 month, 2 months, 3 months, 4 months, 5 months, 6 months, 7 months, 8 months, 9 months, 10 months, 11 months, 12 months. In one embodiment, the subject achieves a partial remission, or a complete remission for 1 year, 2 years, 3 years, or 4 years.

The present embodiments can be understood more fully by reference to the detailed description and examples, which are intended to exemplify non-limiting embodiments.

### EXAMPLES

The examples below were intended to be purely exemplary and should not be considered to be limiting in any way. Unless otherwise specified, the experimental methods in the Examples described below were conventional methods.

### Example 1

TABLE 1
List of Abbreviations and Terms

| Abbreviation | Definition |
|---|---|
| Abs | antibodies |
| ACEI | angiotensin-converting enzyme inhibitor |
| ARB | angiotensin II receptor blockers |
| AUC | Area under plasma concentration-time curve |
| BGB-3111 | zanubrutinib |
| BTK | Bruton tyrosine kinase |
| Cₘₐₓ | maximum plasma concentration |
| CYP | cytochrome P450 |
| DMC | Data Monitoring Committee |
| ECG | electrocardiogram |
| eCRF | electronic case report form |
| EDC | electronic data capture |
| eGFR | estimated glomerular filtration rate |
| EOT | End-of-Treatment (Visit) |
| EQ-5D-5L | European Quality of Life 5-Dimensions |
| | 5-Levels Health Questionnaire |
| ESKD | end-stage kidney disease |
| HBcAb | hepatitis B core antibody |
| HBsAg | hepatitis B surface antigen |
| HBV | hepatitis B virus |
| HCV | hepatitis C virus |
| HRQoL | Health-related quality of life |
| ICF | Informed consent form |
| IEC | Independent Ethics Committee |
| IRB | Institutional Review Board |
| KDQoL-36 | Kidney Disease and Quality |
| | of Life instrument ™-36 items |
| PGI-S | Patient global impression of |
| | symptom severity |
| PK | Pharmacokinetic(s) |
| PLA2R | phospholipase A2 receptor |
| PRO | patient-reported outcome |
| UPCR | urine protein creatinine ratio |

### 1. Study Objectives

### 1.1. Study Objectives

The objectives are to evaluate the efficacy and safety of zanubrutinib in patients with primary membranous nephropathy who are on optimal supportive care.

### Primary:

### Part 1

Evaluate the efficacy of zanubrutinib as measured by proteinuria reduction in patients with primary membranous nephropathy who are on optimal supportive care

### Part 2

Evaluate the efficacy of zanubrutinib compared with tacrolimus as measured by complete remission rate in patients with primary membranous nephropathy who are on optimal supportive care

### Secondary:

### Part 1

Evaluate the efficacy of zanubrutinib in patients with primary membranous nephropathy who are on optimal supportive care as measured by the following:
   Treatment failure
   Immunological response
   Overall remission
   Complete remission
   Relapses
Evaluate the safety and tolerability of zanubrutinib in patients with primary membranous nephropathy

### Part 2

Evaluate the efficacy of zanubrutinib compared with tacrolimus as measured by the following in patients with primary membranous nephropathy who are on optimal supportive care:
   Overall remission
   Treatment failure
   Time to first remission
   Relapses
   Health-related quality of life (HRQoL)
   Prevention of progressive kidney function loss
Evaluate the safety and tolerability of zanubrutinib in patients with primary membranous nephropathy

### Exploratory:

### Part 1

Evaluate the pharmacokinetics (PK) of zanubrutinib
Evaluate the relationship between biomarkers and the efficacy of zanubrutinib

### Part 2

Evaluate the pharmacokinetics (PK) of zanubrutinib
Evaluate immunological response in patients with positive anti-PLA2R Abs at baseline
Evaluate the relationship between biomarkers and the efficacy of zanubrutinib
Severity of patient-reported symptoms

### 1.2. Definition of Primary Estimand

The primary scientific question of interest for Part 2 is as follows: “Will zanubrutinib increase complete remission rate compared with tacrolimus in patients with primary membranous nephropathy who are on optimal supportive care?” The primary estimand is described by the following attributes:
1. Treatments of interest:
The study experimental treatment is zanubrutinib. The study control treatment for comparison is tacrolimus.
2. Population:
All adult patients with primary membranous nephropathy as defined by the inclusion/exclusion criteria.
3. Variable:
The primary variable is complete remission status (Yes/No) at Week 104.
4. Handling of intercurrent events:
Premature discontinuation from the study drug before Week 65: if a patient prematurely discontinues the study drug for any reason (excluding patients who have completed the taper off before Week 65 per protocol in the tacrolimus group), the patient will be considered as a non-responder for complete remission at Week 104 (composite strategy).

Treatment failure: if a patient experiences treatment failure, the patient will be considered as a non-responder for complete remission at Week 104 (composite strategy).
5. Population-level summary:
Complete remission rate difference between zanubrutinib groups and the tacrolimus group at Week 104.

### 2. Study Design

### 2.1. Summary of Study Design

This will be a 2-part, Phase 2/3, multicenter, open-label study to evaluate the efficacy and safety of zanubrutinib in patients with primary membranous nephropathy for a maximum of 132 weeks. Patients who are to be enrolled must have been diagnosed with primary membranous nephropathy according to a renal biopsy. In addition, patients should have urine protein >3.5 g/24 hours at initial screening and at the confirmation assessment.

### 2.1.1. Part 1

Part 1 will be an open-label, single-arm preliminary evaluation of the efficacy and safety of zanubrutinib in patients with primary membranous nephropathy. Thirty eligible patients with anti-PLA2R antibody >50 RU/mL and UPCR (based on 24-hour urine collection, mg/mg or g/g) >3.5 after a 12-week run-in with optimal supportive care, will receive zanubrutinib 160 mg twice a day for 64 weeks.

There will be 4 periods in Part 1: the initial screening period, the run-in period, the treatment period, and the extension observation period, as described below.

Initial screening period: The initial screening assessments will be performed within 4 weeks before the beginning of the 12-week run-in period. Patients who agree to participate will sign the Informed Consent Form (ICF) before any study-specific assessment at screening. Screening procedures are outlined in the Schedule of Assessments (Table 2). Repeated screening procedures or tests are allowed if the patient does not meet the eligibility criteria previously or if the patient needs to have a documented result within the protocol-specified screening window. Re-screening is allowed only once for each patient.

Run-in period: All patients will receive optimal supportive care according to local guidelines during the 12-week run-in period. The maximally tolerated or allowed dose of ACEI or ARB will be given during the run-in period (ACEI or ARB will not be discontinued when there is a modest and stable increase of blood creatinine ≤30%; use of an ACEI with ARB in combination or in combination with a renin inhibitor is prohibited).

If there is any documented record of ACEI or ARB treatment at the maximally tolerated or allowed dose before initiation of the study drug, the duration of treatment would be counted in the run-in period.

In the last 2 weeks of the run-in period, a confirmation assessment will be performed.

Treatment period: The treatment period will be 64 weeks.

After the run-in period, 30 eligible patients will receive zanubrutinib 160 mg twice a day for 64 weeks.

Extension observation period: There will be an extension observation period of 40 weeks after the treatment period. The patients will return for visits as specified in Table 2. Patients who prematurely discontinue the study drug will remain in the study and return for visits as originally planned.

Table 2 depicts the Schedule of Assessments providing the screening procedures of part 1.

TABLE 2
Schedule of Assessments of Part 1

| | Study procedures | | | | | | | | | |
|---|---|---|---|---|---|---|---|---|---|---|
| | Screening and | | | | | | | | | |
| | run-in period ^{p} | | | Treatment period | | | | | | |
| | Initial | Run-in | Confirmation | (±4 days for visits from | | | | | | |
| | Screening | period | assessment | W 1 to W 12, and ±7 days | | | | | | |
| | (−16 W | (−12 W | (−2 W | for the following visits) | | | | | | |
| | to −13 W) | to −D 1) | to −D 1) | W 1 D 1 ^{l} | W 2 | W 4 | W 12 | W 24 | W 36 | W 52 |
| | Day | | | | | | | | | |
| | −112 to −85 | −84 to −1 | −14 to −1 | 1 | 14 | 28 | 84 | 168 | 252 | 364 |
| Screening/ | | | | | | | | | | |
| administrative | | | | | | | | | | |
| Informed consent ^{a} | X | | | | | | | | | |
| Inclusion/ | X | | X | | | | | | | |
| exclusion criteria | | | | | | | | | | |
| Medical history and | X | X | X | | | | | | | |
| demographics | | | | | | | | | | |
| Weight | X | | | X | X | X | X | X | X | X |
| COVID-19 ^{b} | X | | | | | | | | | |
| Renal biopsy ^{c} | X | | | | | | | | | |
| Serum anti-PLA2R | X | | X | | | X | X | X | X | X |
| Abs ^{d} | | | | | | | | | | |
| Patient diary | X | | | | | | | | | |
| dispensation ^{e} | | | | | | | | | | |
| Zanubrutinib | | | | X | | X | X | X | X | X |
| dispensation | | | | | | | | | | |
| Safety assessments | | | | | | | | | | |
| Physical | X | | | X | X | X | X | X | X | X |
| examination ^{f} | | | | | | | | | | |
| HBV and HCV | X | | | | | | | | | |
| HBV monitoring ^{g} | X | | | | | X | X | X | X | X |
| T-SPOT•TB or | X | | | | | | | | | |
| quantiFERON TB | | | | | | | | | | |
| gold test | | | | | | | | | | |
| CD4⁺T cell count | | | X | | | | | | | |
| Pregnancy test ^{h} | X | | X | X | | X | X | X | X | X |
| Vital signs | X | | | X | X | X | X | X | X | X |
| Chest radiographs | X | | | | | | | | | |
| or CT | | | | | | | | | | |
| HbA1c | X | | | | | | | | | |
| Triplicate 12-lead | X | | X | As clinically indicated | | | | | | X |
| ECG ⁱ | | | | | | | | | | |
| Concomitant | X | X | X | X | X | X | X | X | X | X |
| medications and | | | | | | | | | | |
| adverse events ^{k} | | | | | | | | | | |
| Clinical laboratory | | | | | | | | | | |
| assessments | | | | | | | | | | |
| Hematology | X | | X | X | X | X | X | X | X | X |
| Chemistry | X | | X | X | X | X | X | X | X | X |
| Coagulation | X | | | X | | X | X | X | X | X |
| Lipid panel | X | | | X | | | X | X | X | X |
| Serum | X | | | X | | | | X | | X |
| immunoglobulin | | | | | | | | | | |
| Urinalysis ^{l} | X | | | X | X | X | X | X | X | X |
| 24-hour urine | X | | X | | | | X | X | X | X |
| collection ^{m} | | | | | | | | | | |
| Pharmacokinetics ⁿ | See Table 9 | | | | | | | | | |
| Biomarkers ^{o} | | | | | | | | | | |

| | Study procedures | | | | |
|---|---|---|---|---|---|
| | | | | | Safety |
| | Extension observation | | | | Follow-up Visit |
| | period (±7 days) | | | | (±5 days)^{q} |
| | | | | W 104/ | 30 days after |
| | W 65 D 1^{r} | W 76 | W 90 | EOT Visit | the last dose |
| | Day | | | | |
| | 449 | 532 | 630 | 728 | |
| Screening/ | | | | | |
| administrative | | | | | |
| Informed consent ^{a} | | | | | |
| Inclusion/ | | | | | |
| exclusion criteria | | | | | |
| Medical history and | | | | | |
| demographics | | | | | |
| Weight | X | X | X | X | X |
| COVID-19 ^{b} | | | | | |
| Renal biopsy ^{c} | | | | | |
| Serum anti-PLA2R | X | X | X | X | |
| Abs ^{d} | | | | | |
| Patient diary | | | | | |
| dispensation ^{e} | | | | | |
| Zanubrutinib | | | | | |
| dispensation | | | | | |
| Safety assessments | | | | | |
| Physical | X | X | X | X | X |
| examination ^{f} | | | | | |
| HBV and HCV | | | | | |
| HBV monitoring ^{g} | X | X | X | X | |
| T-SPOT•TB or | | | | | |
| quantiFERON TB | | | | | |
| gold test | | | | | |
| CD4⁺T cell count | | | | | |
| Pregnancy test ^{h} | X | X | X | X | X |
| Vital signs | X | X | X | X | X |
| Chest radiographs | | | | | |
| or CT | | | | | |
| HbA1c | | | | | |
| Triplicate 12-lead | As clinically indicated | | | X | X ^{j} |
| ECG ⁱ | | | | | |
| Concomitant | X | X | X | X | X |
| medications and | | | | | |
| adverse events ^{k} | | | | | |
| Clinical laboratory | | | | | |
| assessments | | | | | |
| Hematology | X | X | X | X | X |
| Chemistry | X | X | X | X | X |
| Coagulation | X | X | X | X | |
| Lipid panel | X | X | X | X | |
| Serum | | X | | | |
| immunoglobulin | | | | | |
| Urinalysis ^{l} | X | X | X | X | |
| 24-hour urine | X | X | X | X | |
| collection ^{m} | | | | | |
| Pharmacokinetics ⁿ | See Table 9 | | | | |
| Biomarkers ^{o} | | | | | |

Abbreviations: Abs, antibodies; ACEI, angiotensin-converting enzyme inhibitor; Anti-PLA2R, anti-phospholipase A2 receptor; ARB, angiotensin II receptor blocker; CT, computed tomography; D, day; ECG, electrocardiograms; ELISA, enzyme linked immunosorbent assay; EOT, End-of-Treatment; HbA1c, hemoglobin A1C; HBcAb, hepatitis B core antibody; HBV, hepatitis B virus; HCV, hepatitis C virus; PK, pharmacokinetic(s); TB, tuberculosis; UPCR, urine protein creatinine ratio; W, Week.
^{a} Written informed consent is required before performing any study-specific tests or procedures.
^{b} A COVID-19 test will be conducted according to local practice.
^{c} Renal biopsy before screening is acceptable to confirm eligibility. The renal biopsy results should include light, immunofluorescence, and electron microscopy image descriptions and confirm that the subepithelial deposits are seen on the electron microscopy. If no previous renal biopsy results are available, a renal biopsy must be performed.
^{d} Serum anti-PLA2R Abs will be tested by ELISA for all patients at the screening and the specified visits.
^{e} A patient diary will be dispensed at the initial screening. The patient will record blood pressure and the administration of the study drug at home and bring the diary at each visit for the investigator to review.
^{f} Complete physical examination will be performed at screening and each specified visit, including weight (height at screening only). The body systems will be assessed per standard of care at the study site and as clinically indicated by symptoms.
^{g} Patients with positive anti-HBcAb and HBV DNA < 20 IU/L can be enrolled but need to be monitored for HBV DNA during treatment.
^{h} For women of childbearing potential, a serum pregnancy test must be performed at screening and be documented as negative; a urine pregnancy test will be performed at confirmation assessment and every 4 weeks in the treatment period (a test at home is allowed if no visits are planned during the period). A serum pregnancy test will be performed for confirmation if a urine pregnancy test is positive or equivocal.
ⁱ A 12-lead ECG will be performed in triplicate with the patient in the semirecumbent or supine position at screening, confirmation assessment, at Week 52, at the EOT Visit, and as clinically indicated.
^{j} A 12-lead ECG will be performed as clinically indicated during the Safety Follow-up Visit.
^{k} During the screening and run-in period, only concomitant medications and serious adverse events will be collected. After the initiation of the study drug, all adverse events and serious adverse events, regardless of relationship to the study drug, will be collected until up to 30 days following the last treatment of zanubrutinib, or end of study, whichever comes first. After this period, the investigator should report any serious adverse events that are believed to be related to prior study drug treatment.
^{l} Avoid urine sample collection during menstruation. If urine sample collection cannot be performed because of menstruation, collection could be delayed until the end of menstruation. Unscheduled collection of urine samples should be supplemented within 7 days after menstruation, and this should not be regarded as a protocol deviation.
^{m} The 24-hour urine collection will be used for analyzing UPCR. Measurement includes UPCR and 24-hour urine excretion of protein, creatinine.
ⁿ For details with regard to PK sampling, refer to Table 9
^{o} For details with regard to biomarkers, refer to Table 9
^{p} For patients who have a documented record of ACEI or ARB treatment with a maximally tolerated or allowed dose before the first dose of study treatment, the time window will subtract the treatment duration.
^{q}A patient who prematurely discontinues the study drug and is unwilling to complete subsequent clinic visits will be asked to return for a Safety Follow-up Visit approximately 30 days after the last dose of the study drug.
^{r}The study drug will be discontinued by Week 65 Day 1

### 2.1.2. Part 2

After completing the enrollment of Part 1, the enrollment of Part 2 will start.

Part 2 will be a randomized, open-label, active-controlled evaluation of the efficacy of zanubrutinib compared with tacrolimus and an evaluation of the safety of zanubrutinib in patients with primary membranous nephropathy who are on optimal supportive care. At randomization, eligible patients will be stratified by risk classification of progressive kidney function loss at baseline (moderate risk versus high risk) and region (China versus rest of the world). Approximately 252 eligible patients will be randomized in a 1:1:1 ratio to receive zanubrutinib 160 mg twice a day, 160 mg once a day, or tacrolimus for 64 weeks. Tacrolimus oral capsules with an initial dose of 0.05 mg/kg/day divided into 2 doses will be administered every 12 hours (dose will be adjusted to maintain a target trough blood concentration of 3 to 8 ng/mL) for 52 weeks and then will be tapered off from Week 53, and finally should be discontinued by Week 64.

Patients will be required to receive optimal supportive care during the entire study period. Patients who prematurely discontinue the study drug during the treatment period will remain in the study and return for visits as originally planned. There will be an extension observation period of 40 weeks after the treatment period.

There will be 4 periods in Part 2: the initial screening period, the run-in period, the treatment period, and the extension observation period, as described below.

Initial screening period: The initial screening assessments will be performed within 4 weeks before the beginning of the 24-week run-in period, unless otherwise noted. Patients who agree to participate will sign the Informed Consent Form (ICF) before any study-specific assessment at screening. Screening procedures are outlined in the Schedule of Assessments (Table 3). Repeated screening procedures or tests are allowed if the patient does not meet the eligibility criteria previously or if the patient needs to have a documented result within the protocol-specified screening window. Rescreening is allowed only once for each patient.

Run-in period: All patients will receive optimal supportive care according to local guidelines during the 24-week run-in period, unless otherwise noted. The maximally tolerated or allowed dose of ACEI or ARB will be given during the run-in period (ACEI or ARB will not be discontinued when there is a modest and stable increase of blood creatinine ≤30%; ACEI or ARB combination or combination with a renin inhibitor is prohibited).

If there is any documented record of ACEI or ARB treatment with maximally tolerated or allowed dose before randomization, the duration of treatment would be counted in the run-in period. In the last 2 weeks of the run-in period, a confirmation assessment will be performed. Patients may enter the study and be randomized immediately without the need of the run-in period if they meet either set of the following conditions:

### Set 1 Conditions:

i. Documented evidence of ≥24 weeks of treatment with maximally tolerated or allowed dose of ACEI or ARB with adequate blood pressure control (blood pressure <130/80 mmHg, measured on ≥2 occasions in the month before randomization), AND
ii. Urine protein creatinine ratio (UPCR) (based on 24-hour urine collection) >3.5 at initial screening with a documented <50% reduction during the ≥24 weeks of ACEI or ARB treatment.

### Set 2 Conditions:

i. Adequate blood pressure control (blood pressure <130/80 mmHg, measured on ≥2 occasions in the month before randomization), AND
ii. eGFR<60 mL/min/1.73 m² and/or UPCR (based on 24-hour urine collection) >8 at initial screening.

Randomization and the treatment period: The treatment period will be 64 weeks.

At randomization, eligible patients will be stratified by 2 factors:
Risk classification of progressive kidney function loss at baseline: moderate risk versus high risk
Region: China versus rest of the world

For risk classification of progressive kidney function loss at baseline:

Moderate risk is defined as:
eGFR≥60 mL/min/1.73 m² and UPCR (based on 24-hour urine collection) >3.5 with a documented <50% reduction after 24-week ACEI or ARB treatment; AND
Not fulfilling high-risk criteria.

High risk is defined as:
eGFR<60 mL/min/1.73 m² and/or UPCR (based on 24-hour urine collection) >8; OR
eGFR≥60 mL/min/1.73 m² and UPCR (based on 24-hour urine collection) >3.5 with a documented <50% reduction after 24-week ACEI or ARB treatment; AND at least one of the following: a). serum albumin <25 g/L; b). serum anti-PLA2R Abs>50 RU/mL

These stratification factors were selected to take into account the possible differences in efficacy by region (caused by access to the standard of care and ethnic differences) and risk classification of progressive kidney function loss.

After the run-in period, approximately 252 eligible patients will be randomized in a 1:1:1 ratio to receive zanubrutinib 160 mg twice a day, 160 mg once a day, or tacrolimus for 64 weeks.
Group A: Zanubrutinib 160 mg twice a day
Group B: Zanubrutinib 160 mg once a day
Group C: Tacrolimus oral capsules, with an initial dose of 0.05 mg/kg/day, divided into 2 doses, will be administered every 12 hours for 52 weeks, then will be tapered off from Week 53, and finally should be discontinued by Week 64.

Extension observation period: There will be an extension observation period of 40 weeks after the treatment period. The patients will return for visits as specified in Table 3.

Patients who prematurely discontinue the study drug will remain in the study and return for visits as originally planned.

### Study Assessments:

Assessment of efficacy will include UPCR (based on 24-hour urine collection) and blood creatinine. Patient-reported outcomes (PROs) will be collected using the Kidney Disease and Quality of Life instrument™—36 items (KDQoL-36), European Quality of Life 5-Dimensions 5-Levels Health Questionnaire (EQ-5D-5L), and Patient Global Impression of Symptom Severity (PGI-S) at visits specified in Table 3.

Assessments of safety will include adverse events, serious adverse events, clinical laboratory tests, vital signs, physical examinations, and ECGs. Adverse events will be graded as mild, moderate, or severe. A Data Monitoring Committee (DMC) will monitor the safety data periodically. Blood and urine samples will be collected to evaluate the PK and/or biomarkers as specified in Table 3.

Table 3 depicts the Schedule of Assessments providing the screening procedures of part 2.

TABLE 3
Schedule of Assessments of Part 2

| | Study procedures | | | | | | | | | | |
|---|---|---|---|---|---|---|---|---|---|---|---|
| | Screening and | | | Treatment period | | | | | | | |
| | run-in period ^{p} | | | (±4 days for visits from | | | | | | | |
| | Initial | Run-in | Confirmation | W 1 to W 12, and ±7 days | | | | | | | |
| | Screening | period | assessment | for the following visits) | | | | | | | |
| | (−28 W | (−24 W | (−2 W | W 1 | | | | | | | Optional |
| | to −25 W) | to −D 1) | to −D 1) | D 1 ^{l} | W 2 | W 4 | W 12 | W 24 | W 36 | W 52 | Visits ^{r} |
| | Day | | | | | | | | | | |
| | −196 | −168 | −14 | | | | | | | | |
| | to −176 | to −1 | to −1 | 1 | 14 | 28 | 84 | 168 | 252 | 364 | |
| Screening/ | | | | | | | | | | | |
| administrative | | | | | | | | | | | |
| Informed consent ^{a} | X | | | | | | | | | | |
| Inclusion/ | X | | X | | | | | | | | |
| exclusion criteria | | | | | | | | | | | |
| Medical history and | X | X | X | | | | | | | | |
| demographics | | | | | | | | | | | |
| Weight | X | | | X | X | X | X | X | X | X | X |
| COVID-19 ^{b} | X | | | | | | | | | | |
| Renal biopsy ^{c} | X | | | | | | | | | | |
| Serum anti-PLA2R | X | | X | | | X | X | X | X | X | |
| Abs or | | | | | | | | | | | |
| anti-THSD7A Abs ^{d} | | | | | | | | | | | |
| Randomization | | | | X | | | | | | | |
| Patient diary | X | | | | | | | | | | |
| dispensation ^{e} | | | | | | | | | | | |
| Zanubrutinib | | | | X | | X | X | X | X | X | |
| dispensation | | | | | | | | | | | |
| Tacrolimus | | | | X | X | X | X | X | X | X | X |
| dispensation | | | | | | | | | | | |
| Safety assessments | | | | | | | | | | | |
| Physical | X | | | X | X | X | X | X | X | X | |
| examination ^{f} | | | | | | | | | | | |
| HBV and HCV | X | | | | | | | | | | |
| HBV monitoring ^{g} | X | | | | | X | X | X | X | X | |
| T-SPOT•TB or | X | | | | | | | | | | |
| quantiFERON TB | | | | | | | | | | | |
| gold test | | | | | | | | | | | |
| CD4⁺T cell count | | | X | | | | | | | | |
| Pregnancy test ^{h} | X | | X | X | | X | X | X | X | X | |
| Vital signs | X | | | X | X | X | X | X | X | X | X |
| Chest radiographs or | X | | | | | | | | | | |
| CT | | | | | | | | | | | |
| HbA1c | X | | | X | | | X | X | X | X | |
| Triplicate 12-lead | X | | X | As clinically indicated | | | | | | X | As clinically |
| ECG ⁱ | | | | | | | | | | | indicated |
| Concomitant | X | X | X | X | X | X | X | X | X | X | X |
| medications and | | | | | | | | | | | |
| adverse events ^{k} | | | | | | | | | | | |
| PRO | | | | | | | | | | | |
| KDQoL-36 | | | | X | | | | X | | X | |
| EQ-5D-5L | | | | X | | | | X | | X | |
| questionnaire | | | | | | | | | | | |
| PGI-S | | | | X | | | | X | | X | |
| Clinical laboratory | | | | | | | | | | | |
| assessments | | | | | | | | | | | |
| Hematology | X | | X | X | X | X | X | X | X | X | X |
| Chemistry | X | | X | X | X | X | X | X | X | X | X |
| Coagulation | X | | | X | | X | X | X | X | X | |
| Lipid panel | X | | | X | | | X | X | X | X | |
| Serum | X | | | X | | | | X | | X | |
| immunoglobulins | | | | | | | | | | | |
| Urinalysis ^{l} | X | | | X | X | X | X | X | X | X | |
| 24-hour urine | X | | X | | | | X | X | X | X | |
| collection ^{m} | | | | | | | | | | | |
| Tacrolimus trough | | | | | X | X | X | X | X | X | X |
| concentration | | | | | | | | | | | |
| Pharmacokinetics ⁿ | See Table 9 | | | | | | | | | | |
| Biomarkers ^{o} | | | | | | | | | | | |

| | Study procedures | | | | |
|---|---|---|---|---|---|
| | | | | | Safety |
| | Extension observation | | | | Follow-up Visit |
| | period (±7 days) | | | | (±5 days) ^{q} |
| | | | | W 104/ | 30 days after |
| | W 65 D 1 ^{s} | W 76 | W 90 | EOT Visit | the last dose |
| | Day | | | | |
| | 449 | 532 | 630 | 728 | |
| Screening/ | | | | | |
| administrative | | | | | |
| Informed consent ^{a} | | | | | |
| Inclusion/ | | | | | |
| exclusion criteria | | | | | |
| Medical history and | | | | | |
| demographics | | | | | |
| Weight | X | X | X | X | X |
| COVID-19 ^{b} | | | | | |
| Renal biopsy ^{c} | | | | | |
| Serum anti-PLA2R | X | X | X | X | |
| Abs or | | | | | |
| anti-THSD7A Abs ^{d} | | | | | |
| Randomization | | | | | |
| Patient diary | | | | | |
| dispensation ^{e} | | | | | |
| Zanubrutinib | | | | | |
| dispensation | | | | | |
| Tacrolimus | | | | | |
| dispensation | | | | | |
| Safety assessments | | | | | |
| Physical | X | X | X | X | X |
| examination ^{f} | | | | | |
| HBV and HCV | | | | | |
| HBV monitoring ^{g} | X | X | X | X | |
| T-SPOT•TB or | | | | | |
| quantiFERON TB | | | | | |
| gold test | | | | | |
| CD4⁺T cell count | | | | | |
| Pregnancy test ^{h} | X | X | X | X | X |
| Vital signs | X | X | X | X | X |
| Chest radiographs or | | | | | |
| CT | | | | | |
| HbA1c | X | X | X | X | X |
| Triplicate 12-lead | As clinically indicated | | | X | X ^{j} |
| ECG ⁱ | | | | | |
| Concomitant | X | X | X | X | X |
| medications and | | | | | |
| adverse events ^{k} | | | | | |
| PRO | | | | | |
| KDQoL-36 | | X | | X | |
| EQ-5D-5L | | X | | X | |
| questionnaire | | | | | |
| PGI-S | | | | | |
| Clinical laboratory | | | | | |
| assessments | | | | | |
| Hematology | X | X | X | X | X |
| Chemistry | X | X | X | X | X |
| Coagulation | X | X | X | X | |
| Lipid panel | X | X | X | X | |
| Serum | | X | | | |
| immunoglobulins | | | | | |
| Urinalysis ^{l} | X | X | X | X | |
| 24-hour urine | X | X | X | X | |
| collection ^{m} | | | | | |
| Tacrolimus trough | | | | | |
| concentration | | | | | |
| Pharmacokinetics ⁿ | See Table 9 | | | | |
| Biomarkers ^{o} | | | | | |

Abbreviations: Abs, antibodies; Anti-PLA2R, anti-phospholipase A2 receptor; CT, computed tomography; D, day; ECG, electrocardiograms; ELISA, enzyme linked immunosorbent assay; EOT, End-of-Treatment; EQ-5D-5L, European Quality of Life 5-Dimensions 5-Levels Health Questionnaire; HbA1c, hemoglobin A1C; HBcAb, hepatitis B core antibody; HBV, hepatitis B virus; HCV, hepatitis C virus; KDQoL-36, Kidney Disease and Quality of Life instrument ™ - 36 items; PGI-S, Patient Global Impression of Symptom Severity; PK, pharmacokinetic(s); PRO, patient-reported outcome; TB, tuberculosis; THSD7A, thrombospondin type 1 domain containing 7A; UPCR, urine protein creatinine ratio; W, Week.
^{a} Written informed consent is required before performing any study-specific tests or procedures.
^{b} A COVID-19 test will be conducted according to local practice.
^{c} Renal biopsy before screening is acceptable to confirm eligibility. The renal biopsy results should include light, immunofluorescence, and electron microscopy image descriptions and confirm that the subepithelial deposits are seen on the electron microscopy. If no previous renal biopsy results are available, a renal biopsy must be performed.
^{d} Serum anti-PLA2R Abs will be tested by ELISA for all patients at the screening and for all patients with baseline positive anti-PLA2R Abs before the administration of the study drug at the specified visits. For the patients with negative anti-PLA2R Abs at initial screening, a test of anti-THSD7A Abs will be performed at confirmation assessment and then the following visit if the result is positive at baseline.
^{e} A patient diary will be dispensed at the initial screening. The patient will record blood pressure and the administration of the study drug at home and bring the diary at each visit for the investigator to review.
^{f} Complete physical examination will be performed at screening and each specified visit, including weight (height at screening only). The body systems will be assessed per standard of care at the study site and as clinically indicated by symptoms.
^{g} Patients with positive anti-HbcAb and HBV DNA <20 IU/L can be enrolled but need to be monitored for HBV DNA during treatment.
^{h} For women of childbearing potential, a serum pregnancy test must be performed at screening and be documented as negative; a urine pregnancy test will be performed at confirmation assessment and every 4 weeks in the treatment period (a test at home is allowed if no visits are planned during the period). A serum pregnancy test will be performed for confirmation if a urine pregnancy test is positive or equivocal.
ⁱ A 12-lead ECG will be performed in triplicate with the patient in the semirecumbent or supine position at screening, confirmation assessment, at Week 52, at the EOT Visit, and as clinically indicated.
^{j} A 12-lead ECG will be performed as clinically indicated during the Safety Follow-up Visit.
^{k} During the screening and run-in period, only concomitant medications and serious adverse events will be collected. After the initiation of the study drug, all adverse events and serious adverse events, regardless of relationship to the study drug, will be collected until up to 30 days following the last treatment of zanubrutinib or tacrolimus, or end of study, whichever comes first. After this period, the investigator should report any serious adverse events that are believed to be related to prior study drug treatment.
^{l} Avoid urine sample collection during menstruation. If urine sample collection cannot be performed because of menstruation, collection could be delayed until the end of menstruation. Unscheduled collection of urine samples should be supplemented within 7 days after menstruation, and this should not be regarded as a protocol deviation.
^{m} The 24-hour urine collection will be used for analyzing UPCR. Measurement includes UPCR and 24 hours urine excretion of protein, creatinine.
ⁿ For details with regard to PK sampling, refer to Table 9.
^{o} For details with regard to biomarkers, refer to Table 9.
^{p} For patients who have a documented record of ACEI or ARB treatment with a maximally tolerated or allowed dose before randomization, the time window will subtract the treatment duration. For the patients who could skip the run-in period, screening assessments will combine with confirmatory assessments.
^{q} A patient who prematurely discontinues the study drug and is unwilling to complete more clinic visits will be asked to return for a Safety Follow-up Visit approximately 30 days after the last dose of the study drug.
^{r} For patients in Group C, in order to adjust the tacrolimus dose, there may be optional visits to retest blood drug concentration, serum creatinine, serum potassium, fasting blood glucose etc at the discretion of the investigator.
^{s} The study drug will be discontinued by Week 65 Day 1.

### 2.1.3. Rescue Therapy

Rescue therapy will be allowed for both Part 1 and Part 2 but not mandatory when any of the following conditions occurs and secondary causes have been excluded:
At Week 24, reduction in urine protein creatinine ratio (UPCR) (based on 24-hour urine collection) is <25% compared with the baseline (confirmed with a repeated measurement within 2 weeks), UPCR (based on 24-hour urine collection) >3.5, AND, for patients who had positive anti-PLA2R Abs at baseline, antibody level >50 RU/mL.
At Week 52, reduction in UPCR (based on 24-hour urine collection) is <50% compared with the baseline (confirmed with a repeated measurement within 2 weeks), UPCR (based on 24-hour urine collection) >3.5, AND, for patients who had positive anti-PLA2R Abs at baseline, where immunological response is not achieved.
From Week 65, in patients with relapses.
   Rescue therapy includes the following:
Rituximab: a dose of rituximab 1 g by intravenous infusion, 2 weeks apart, OR
Cyclophosphamide+glucocorticoids Note: Dosage and duration of the rescue therapy could refer to local treatment guidelines.

If a patient receives any rescue therapy, the study drug should be discontinued.

### 2.2. Study Schema

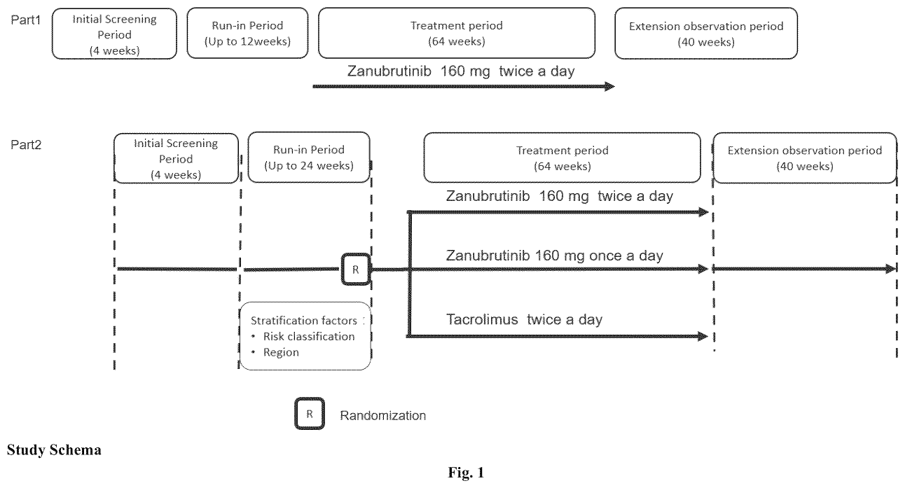

The study schema is provided in FIG. 1.

### 2.3. Duration of Study

The total duration of this study is expected to be approximately 5.5 years.

### 2.4. Discussion of Study Design, Including Choice of Control Group

This study is designed to test whether treatment with zanubrutinib is effective and tolerant in patients with primary membranous nephropathy who are on optimal supportive care.

### Two-Part Study Design

Part 1 will be an open-label, single arm, preliminary investigation of the efficacy and safety of zanubrutinib in patients with primary membranous nephropathy who are on optimal supportive care. The primary endpoint in this part is the change of proteinuria at Week 24 compared with baseline. The safety evaluation and several key efficacy endpoints, like the proportion of patients with treatment failure and immunological response at Week 24, will be descriptively analyzed.

Part 2 is a Phase 3, randomized, multicenter, active-controlled, open-label evaluation of the efficacy and safety of zanubrutinib in patients with primary membranous nephropathy. Patients who are to be enrolled should have been treated with optimal supportive care for at least 24 weeks unless otherwise noted. In addition, patients should have UPCR (based on 24-hour urine collection) >3.5 at initial screening and at the confirmation assessment. Two different regimens of zanubrutinib will be compared with the active control. The primary endpoint of Part 2 is complete remission status (Yes/No) at Week 104.

Based on the efficacy and safety results at Week 24 in Part 1, including proportion of patients with treatment failure and immunological response, the study may be terminated in case of unsatisfactory efficacy and/or safety results.

### Selection of Endpoints

The primary efficacy endpoint of clinical trials for treatment of chronic kidney disease ideally should be the delay of progressive kidney function loss before reaching ESKD. However, patients with primary membranous nephropathy are likely to experience progressive kidney function loss over many years before reaching ESKD, a timeframe that is not feasible for the conduct of clinical trials. Using remission of proteinuria as a surrogate endpoint facilitates the conduct of clinical studies for patients with chronic kidney disease. Complete remission of proteinuria has been evaluated and has been shown to be associated with excellent long-term renal survival rates. In a cohort study of patients with primary membranous nephropathy from the Toronto Glomerulonephritis Registry, none of the patients who achieved complete remission progressed to ESKD over a median follow-up of 87 months (14 to 400 months). Results demonstrated the mean slope of decline in creatinine clearance was −0.12±0.4 mL/min/month. In a Spanish cohort study with a large population of 328 patients with primary membranous nephropathy, long-term renal survival (over a mean follow-up of 104 months) in patients with complete remission demonstrated that complete remission has a favorable long-term prognosis. Several available analyses of clinical study data suggest that treatment effects on complete remission at 1 and 2 years may predict a treatment's effect on long-term renal survival. Another cohort study reported that the cumulative probability of patients with complete remission reaching the primary outcome (defined as ESKD or 50% reduction in eGFR) was significantly lower than in either the partial remission or relapse cohort from the 3-month to the 24-month landmarks, which support that complete remission could be an acceptable endpoint for clinical trials.

### Dose Selection Rationale

This study will evaluate the efficacy and safety of 2 different regimens of zanubrutinib in patients with primary membranous nephropathy. The dose regimen of 320 mg per day (160 mg twice a day) selected as the high dose for this study is consistent with the approved clinical dose in hematologic indications, which has been shown to be efficacious and well tolerated in patients with B-cell malignancies. According to the studies of BTK inhibitors in patients with autoimmune diseases, the efficacious dose should achieve sustained BTK occupancy >90% in PBMCs and >70% in the spleen. A total daily dose of 160 mg once a day resulted in BTK occupancy >75% in all PBMCs with the median BTK occupancy approached 100% at each timepoint post dosing. More importantly, once a day administration has an advantage over twice a day in terms of compliance among patients with chronic diseases; therefore, the low dose of 160 mg once a day will be included. In summary, the efficacy and safety of zanubrutinib at 160 mg twice a day and 160 mg once a day will be explored in this study.

### Choice of the Control Group

Considering the possible spontaneous remission of membranous nephropathy and the side effects of immunosuppressive therapy, the updated KDIGO 2021 guideline recommends conservative therapy for patients with a low or moderate risk of progressive kidney function loss. The effect of optimal ACEI or ARB therapy is usually observed within 2 months of initiation of the medication and tends to be modest with a 40% to 50% reduction in proteinuria. However, patients with a pre-existing high level of proteinuria or a high level of anti-PLA2R Abs are less likely to achieve the complete remission target using conservative therapies alone, even when these medications are used at the highest dose. In clinical studies of long-term treatment duration, it is unethical to select the conservative therapy treatments as a control for patients who need immunosuppressive therapy. Alkylating agents, such as cyclophosphamide combined with glucocorticoids, are toxic drugs with severe short-term and long-term adverse effects, despite strong evidence of their efficacy in increasing remission and preventing the loss of kidney function. Therefore, the cyclophosphamide-based immunosuppressive therapy is restricted to patients with a very high risk of rapid deterioration of kidney function and patients with life-threatening nephrotic syndrome. This study intends to target a patient population who fulfill the criteria for moderate to high risk of progressive kidney function loss as defined in the KDIGO 2021 Guideline and are unlikely to have a favorable clinical course after conservative treatment, ie, with nephrotic proteinuria and <50% reduction in 24-hour urine protein after run-in. Tacrolimus monotherapy has been shown to be effective in inducing complete and partial remission in RCTs and cohort studies, and its beneficial side-effect profile favors its use over cyclophosphamide as an initial treatment in patients with primary membranous nephropathy at moderate to high risks of progressive kidney function loss. It is reasonable to choose tacrolimus as a control drug in this study. Although the high relapse rate after treatment with calcineurin inhibitors is a cause for concern, rescue therapy for that condition will be available in this study for the benefit of the enrolled patients.

### 3. Selection of Study Population

The specific eligibility criteria for selection of patients are provided in Section 3.1 and Section 3.2. The sponsor will not grant any eligibility waivers. All laboratory assessments will be performed by a qualified local laboratory unless otherwise specified.

### 3.1. Inclusion Criteria

### 3.1.1. Part 1 and Part 2

Each patient eligible to participate in Part 1 and Part 2 must meet all of the following criteria:
1. 18 to 75 years of age (inclusive) on the day of signing the ICF.
2. Be able to provide written informed consent (by the patient or by the patient's legally acceptable representative) and understand and agree to comply with the requirements of the study and the Schedule of Assessments.
3. Biopsy-confirmed primary membranous nephropathy.
4. UPCR (based on 24-hour urine collection) >3.5 at the initial screening and at the confirmation assessment.
5. Female patients of childbearing potential must practice highly effective methods of contraception (Section 4.3) from 4 weeks before the first dose of study drug, for the duration of the study, and for ≥30 days after the last dose of the study drug.

Male patients are eligible if they are abstinent (defined as refraining from heterosexual intercourse during the entire period of exposure associated with the study drug, starting the day before the first dose of study drug, for the duration of the study, and for ≥30 days after the last dose of the study drug), vasectomized, or if they agree to use barrier contraception in combination with other methods described in Section 4.3 during the study treatment period and for ≥30 days after the last dose of the study drug.

### 3.1.2. Part 1 Only

Each patient eligible to participate in Part 1 must meet all the criteria in Section 3.1.1 and the following criteria:
1. Treatment with maximally tolerated or allowed dose of ACEI or ARB for ≥12 weeks before the first dose of zanubrutinib and with adequate blood pressure control (blood pressure <130/80 mmHg, measured on ≥2 occasions in the month before the assignment of study treatment).
2. Anti-PLA2R antibody >50 RU/mL at confirmation assessment

### 3.1.3. Part 2 Only

Each patient eligible to participate in Part 2 must meet all the criteria in Section 3.1.1 and the following criterion:

1. Treatment with maximally tolerated or allowed dose of ACEI or ARB for ≥24 weeks before randomization and with adequate blood pressure control (blood pressure <130/80 mmHg, measured on ≥2 occasions in the month before randomization). This criterion can be exempted for patients with Set 2 conditions below and patients will be randomized without the run-in period if they have either one of the 2 sets of conditions below:

### Set 1 Conditions:

i. Documented evidence of ≥24 weeks of treatment with maximally tolerated or allowed dose of ACEI or ARB with adequate blood pressure control (blood pressure <130/80 mmHg, measured on ≥2 occasions in the month before randomization), AND
ii. UPCR (based on 24-hour urine collection) >3.5 at initial screening with a documented <50% reduction during the ≥24 weeks of ACEI or ARB treatment

### Set 2 Conditions:

i. Adequate blood pressure control (blood pressure <130/80 mmHg, measured on ≥2 occasions in the month before randomization), AND
ii. eGFR<60 mL/min/1.73 m² and/or UPCR (based on 24-hour urine collection) >8 at initial screening.

### 3.2. Exclusion Criteria

### 3.2.1. Part 1 and Part 2

Each patient eligible to participate in Part 1 and Part 2 must NOT meet any of the following exclusion criteria:
1. Lactating or pregnant female patients.
2. Patients with a secondary cause of membranous nephropathy (eg, diabetic nephropathy, lupus nephritis) or with other primary glomerular diseases, such as Immunoglobulin A (IgA) nephropathy, etc.
3. Type 1 or 2 diabetes mellitus with hemoglobin Alc (HbAlc)≥7% at screening.
4. The eGFR<40 mL/min/1.73 m² (according to the CKD-EPI formula), or initiation of dialysis.
5. Ongoing treatment with a strong CYP3A inhibitor or inducer (refer to Table).
6. Prior exposure to a BTK inhibitor.
7. Receipt of any prohibited drug within specified time (refer to Table and Table).
8. A known history of a primary immunodeficiency or an underlying condition such as human immunodeficiency virus (HIV) infection or splenectomy that predisposes the patient to infections.
9. Positive tuberculosis at screening:
   a. Has a history of latent or active tuberculosis before the assignment of study treatment. An exception is made for patients with a history of latent tuberculosis and documentation of having completed appropriate treatment for latent tuberculosis before the first dose of study drug. It is the responsibility of the investigator to verify the adequacy of previous anti-tuberculosis treatment and provide appropriate documentation.
      Note: Tuberculosis preventive treatment options include 6 months of daily isoniazid, a 3-month regimen of weekly rifapentine plus isoniazid, or a 3-month regimen of daily isoniazid plus rifampicin. A 1-month regimen of daily rifapentine plus isoniazid or 4 months of daily rifampicin alone may also be offered as alternatives.
   b. Has signs or symptoms suggestive of active tuberculosis according to medical history and/or physical examination during screening.
   c. Has had recent close contact with a person with active tuberculosis or, if there has been such contact, will be referred to a physician specialized in tuberculosis to undergo additional evaluation and, if warranted, receive appropriate treatment.
      Note: Appropriate treatment is the same as the preventive treatment options defined in the note for Criterion a.
   d. Has a positive interferon-gamma release assay screening test result (T-SPOT.TB [Oxford Immunotec] or quantiFERON-TB Gold test will be used in this study). A patient whose initial interferon gamma release assay test result is ambiguous should have the test repeated while still fulfilling the other tuberculosis criteria for inclusion. The test should not be repeated if other risk factors for tuberculosis are present. If the test is again ambiguous, the patient will be excluded. In case of a positive interferon gamma release assay test result due to previous latent tuberculosis, the patient is eligible if adequate documentation of completed antituberculosis treatment before randomization is available.
   e. Has a chest radiograph or computed tomography, read by a qualified radiologist, whose diagnostic assessment is consistent with evidence of current active tuberculosis or old, inactive tuberculosis, taken within 12 weeks before the assignment of study treatment.
10. Active granulomatous infection before the assignment of study treatment; nontuberculous mycobacterial infection or opportunistic infection requiring hospitalization or parenteral antimicrobial treatment within 1 year before the assignment of study treatment.
11. Any infections requiring hospitalization or treatment with intravenous anti-infectives not completed ≥4 weeks before the assignment of study treatment.
12. Severe hepatic insufficiency (Child-Pugh C).
13. Current alcohol, drug, or chemical abuse, or a history of such abuse within 1 year before the assignment of study treatment.
14. History of a cancer, apart from cervical cancer in situ, basal or squamous cell skin cancer treated with curative therapy ≥2 year prior to the assignment of study treatment.
15. History of intracranial hemorrhage.
16. Clinically significant cardio-cerebrovascular diseases including the following:
   a. Unstable angina, or myocardial or cerebral infarction within 9 months before the assignment of study treatment
   b. New York Heart Association class III or IV congestive heart failure in the following table.

| NYHA Class | Symptoms |
|---|---|
| I | Cardiac disease, but no symptoms and no limitation |
| | in ordinary physical activity, eg, no shortness of |
| | breath when walking, climbing stairs, etc. |
| II | Mild symptoms (mild shortness of breath and/ |
| | or angina) and slight limitation during ordinary activity. |
| III | Marked limitation in activity due to symptoms, even |
| | during less-than-ordinary activity, eg, walking short |
| | distances (20-100 m). Comfortable only at rest. |
| IV | Severe limitations. Experiences symptoms even |
| | while at rest. Mostly bedbound patients. |

c. History of clinically significant arrhythmias (eg, sustained ventricular tachycardia, ventricular fibrillation, torsades de pointes)
d. QTcF>480 msec based on Fredericia's formula
e. History of Mobitz II second-degree or third-degree heart block without a permanent pacemaker in place
f Hypertension as indicated by a minimum of 2 consecutive blood pressure measurements showing systolic blood pressure >130 mmHg and diastolic blood pressure >80 mmHg in the last month before the assignment of study treatment
17. History of severe bleeding disorder such as hemophilia A, hemophilia B, and von Willebrand disease, or history of spontaneous bleeding requiring blood transfusion or other medical intervention.
18. Unable to swallow capsules or disease significantly affecting gastrointestinal function such as malabsorption syndrome, resection of the stomach or small bowel, bariatric surgery procedures, symptomatic inflammatory bowel disease, or partial or complete bowel obstruction.
19. Known infection with serologic status reflecting active or chronic HBV, or presence of hepatitis C virus (HCV) antibody as follows:
   a. Presence of hepatitis B surface antigen (HBsAg) or hepatitis B core antibody (HBcAb). Patients with presence of HBcAb, but absence of HBsAg, are eligible if HBV DNA is undetectable (<20 IU/mL), and if they are willing to undergo monitoring for HBV reactivation.
   b. Presence of HCV antibody.
20. Major surgery within 4 weeks before the assignment of study treatment.
21. Vaccination with a live vaccine within 4 weeks before the assignment of study treatment.
Note: Vaccines for COVID-19 are allowed except for any live vaccine that may be developed. Seasonal vaccines for influenza are generally inactivated vaccines and are allowed. Intranasal vaccines are live vaccines and are not allowed.
22. Concurrent participation in another therapeutic clinical trial.
23. Positive test result for COVID-19 as determined by antigen testing or polymerase chain reaction (PCR) testing by a licensed method within 4 weeks before the assignment of study treatment.
24. Unwilling or unable to participate in all required study evaluations and procedures.
25. Unwilling or unable to follow the dietary guidance per investigator.
26. Unable to understand the purpose and risks of the study and to provide a signed and dated ICF and authorization to use protected health information (in accordance with national and local patient privacy regulations), except to the extent that surrogate consents and assents are obtained in accordance with national and local laws and regulations. For clarity, the surrogate consents and assents may include, but are not limited to the following: court-appointed guardians, healthcare proxies, durable powers of attorney, or family members/next-of-kin.
27. Any conditions that in the opinion of the investigator, will either alone or in combination:
   a. Render the administration of study drug hazardous.
   b. Interfere with evaluation of the investigational product.
   c. Interfere with interpretation of patient safety or study results.
28. Alanine aminotransferase and/or aspartate aminotransferase ≥3× the upper limit of normal (ULN) and total bilirubin ≥2×ULN; and, in the absence of colony-stimulating factor or blood transfusion, all of the following within 14 days before the assignment of study treatment:
   hemoglobin≤80 g/L (8.0 g/dL)
   platelet count ≤50×10⁹/L (50,000 cells/mm³)
   white blood cell count ≤2.0×10⁹/L (2,000 cells/mm³)
   neutrophils ≤1.5×10⁹/L (1500 cells/mm³)
   CD4+ T cell count ≤0.4×10⁹/L (400 cells/mm³)
Note: If there is any abnormal screening laboratory test, the test may be repeated ONCE on a separate sample before the patient is declared as a screen failure

### 3.2.2. Part 1 Only

Each patient eligible to participate in Part 1 must NOT meet any of the exclusion criteria in Section 3.2.1 and the following criteria:
1. Hypersensitivity to zanubrutinib, or its formulation excipients
2. Evidence of ≥50% reduction in 24-hour urine protein within 12 weeks before the assignment of zanubrutinib.

### 3.2.3. Part 2 Only

Each patient eligible to participate in Part 2 must NOT meet any of the exclusion criteria in Section 3.2.1 and the following criteria:
1 Evidence of ≥50% reduction in 24-hour urine protein within 24 weeks before randomization
2 History of resistance or intolerance to tacrolimus.
3 Hypersensitivity to zanubrutinib, tacrolimus, or their formulation excipients.

### 4 Enrollment and Study Procedures

Study enrolment and procedures are summarized in the following subsections. The timing of all study procedures is provided in the Schedule of Assessments (Table 2 and Table 3).

### 4.1 Visit Windows

A study visit is scheduled for some specified study weeks (Table 2 and Table 3). Procedures for a given visit may be split across the window to allow for drug resupply and completion of study procedures.

### 4.2 Informed Consent

At the Screening Visit, study site personnel must explain to potential study participants all aspects of the study, including all scheduled visits and activities. A copy of the ICF will be given to the patient to read and the patient must have adequate time to fully understand the study and to ask questions. Study site personnel must obtain signed informed consent before any study-specific procedures are conducted unless the procedures are part of optimal supportive care and must document the informed consent process in the patient's clinical record. Informed consent will be obtained before the screening period. Consent must be obtained using the most current version of the form approved by the Independent Ethics Committee/Institutional Review Board (IEC/IRB).

Repeated screening procedures or tests are allowed if the patient dose not previously meet the eligibility criteria or if the patient needs to have a documented result within the protocol-specified screening window. Re-screening is allowed only once for each patient.

For patients who provide informed consent and subsequently do not meet eligibility criteria or who withdraw consent before randomization, study site personnel should document the screen failure in the patients' source documents. The documentation should include demographics and medical history, the reason for screen failure, the eligibility criteria reviewed, procedures performed, etc.

### 4.3 Women of Childbearing Potential and Contraception

A woman is considered of childbearing potential (ie, fertile) following menarche and until becoming post-menopausal unless permanently sterile. Permanent sterilization methods include hysterectomy, bilateral salpingectomy, and bilateral oophorectomy. Contraception methods include the following:
Combined (estrogen- and progestogen containing) hormonal contraception associated with the inhibition of ovulation (oral, intravaginal, or transdermal)
Progestogen-only hormonal contraception associated with the inhibition of ovulation (oral, injectable, or implantable)
An intrauterine device
Intrauterine hormone-releasing system
Bilateral tubal occlusion
Vasectomized partner, provided that the vasectomized partner is the sole sexual partner of the woman of childbearing potential study participant and that the vasectomized partner has received medical assessment of surgical success
Sexual abstinence, defined as refraining from heterosexual intercourse during the entire period of risk associated with the study treatment, starting the day before the first dose of study drug, for the duration of the study, and for ≥30 days after the last dose of zanubrutinib or tacrolimus. Total sexual abstinence should only be used as a contraceptive method if it is in line with the patient's usual and preferred lifestyle. Periodic abstinence (eg, calendar, ovulation, sympto-thermal, post-ovulation methods), declaration of abstinence for the duration of exposure to investigational medicinal product, and withdrawal are not acceptable methods of contraception.

Of note, barrier contraception (including male and female condoms with or without spermicide) is not considered a highly effective method of contraception, and, if used, this method must be used in combination with another acceptable method listed above.

Patients using hormonal contraceptives (eg, birth control pills or devices) must use a barrier method of contraception (eg, condoms) as well.

A post-menopausal state is defined as no menses for 12 months without an alternative medical cause. A high follicle-stimulating hormone level in the post-menopausal range may be used to confirm a post-menopausal state in women not using hormonal contraception or hormonal replacement therapy. However, in the absence of 12 months of amenorrhea, a single follicle-stimulating hormone measurement is insufficient.

### 4.4 Enrollment and Randomization

The initial screening assessments must be performed within 4 weeks before the run-in period unless otherwise noted. There will be a confirmation assessment in the last 2 weeks of the run-in period. The site investigator is responsible for maintaining a record of all patients screened and those who are enrolled in the study. Screening procedures are outlined in the Schedule of Assessments (Table 2 and Table 3).

### 4.4.1 Patient Numbering

After obtaining informed consent, study site personnel will access the Interactive Response Technology (IRT) system to assign a unique patient number to a potential study participant. Patient numbers will be assigned in chronological order starting with the lowest number. Once a patient number has been assigned to a patient, it cannot be reassigned to any other patient.

4.4.2 Run-in Period All patients will receive optimal supportive care during the run-in period unless otherwise noted. The maximally tolerated or allowed dose of ACEI or ARB will be given during the run-in period. Blood pressure should be monitored during the run-in period and be confirmed as <130/80 mmHg, measured on ≥2 occasions in the last month of the run-in period before the assignment of study treatment.

If there is any documented record of ACEI or ARB treatment with maximally tolerated or allowed dose before the first dose of study drug, the duration of treatment would be calculated in the run-in period.

In Part 2, patients may enter the study and be randomized immediately without the need of the run-in period if they meet either set of the following conditions:

### Set 1 Conditions:

i. Documented evidence of ≥24 weeks of treatment with maximally tolerated or allowed dose of ACEI or ARB with adequate blood pressure control (blood pressure <130/80 mmHg, measured on ≥2 occasions in the month before randomization), AND
ii. UPCR (based on 24-hour urine collection) >3.5 at initial screening with a documented <50% reduction during the ≥24 weeks of ACEI or ARB treatment, AND

### Set 2 Conditions:

i. Adequate blood pressure control (blood pressure <130/80 mmHg, measured on ≥2 occasions in the month before randomization), AND
ii. eGFR<60 mL/min/1.73 m² and/or UPCR (based on 24-hour urine collection) >8 at initial screening.

### 4.4.3 Medical History

During screening, review of any medical history any time will start after obtaining informed consent. Clinically significant medical history findings (ie, previous diagnoses, diseases, or surgeries) not pertaining to the study indication, started before signing the informed consent, and considered relevant for the patient's study eligibility will be collected and captured in the electronic case report form (eCRF). “Clinically significant” is defined as any events, diagnoses or laboratory values requiring treatment, or follow-up or the presence of signs or symptoms that require medical intervention. Clinically significant concurrent medical signs and symptoms during the screening period must be documented to establish baseline severities.

Prior medications/significant non-drug therapies and demographic data will also be collected. Patient demographics include full date (only if required and permitted) or year of birth or age, sex, race or predominant ethnicity (if permitted), smoking history, and relevant medical history or current medical conditions (until date of signature of informed consent), all of which will be recorded in the eCRF. Patient race or ethnicity data are collected and analyzed to identify any differences in the safety and/or efficacy profile of the treatment due to these characteristics. In addition, these data are necessary to assess the diversity of the study population as required by health authorities.

Information will also be collected regarding childbearing potential and any other assessments that are done for the purpose of eligibility for inclusion into the study (eg, physical examination, vital signs, hematology and blood chemistry, urinalysis, pregnancy test, and ECG). For further details on eligibility assessments, please see Section 3.

### 4.4.4 Confirmation of Eligibility

Prior to enrollment, the investigator is responsible for assessing and confirming that each patient meets all inclusion eligibility criteria for this study and that none of the exclusion criteria apply. All results from the screening procedures and relevant medical history must be available and reviewed by the investigator before eligibility can be determined. No eligibility waivers will be granted. Sponsor verification of patient eligibility will be managed by way of source data verification in accordance with International Council for Harmonisation (ICH) E6.

The sponsor's medical monitor will support the investigator and/or site staff by answering any queries or questions relating to protocol eligibility criteria.

### 4.4.5 Enrollment/Randomization

In Part 1, patients will be enrolled and assigned the study drug via IRT when the investigator confirms eligibility. When all 30 patients have been enrolled in the study, the enrollment of Part 2 will be started.

In Part 2, site personnel will access the IRT system to randomly assign the patient to a treatment group and to assign the study drug. The study drug must commence ≤1 day after randomization. At randomization, patients will be stratified by risk classification of progressive kidney function loss at baseline (moderate risk versus high risk) and region (China versus rest of the world [ROW]).

### 4.5 Study Drug Dispensation

The study drug (zanubrutinib or tacrolimus) will be dispensed by the study site personnel to patients at scheduled study visits to ensure an adequate drug supply for administration throughout the treatment period as detailed in the Pharmacy Manual. Instructions are to be provided for dosing, storage, and return of all packs (used and unused) at future visits.

### 4.6 Pharmacokinetics

Blood samples will be collected to characterize the PK of zanubrutinib in patients with primary membranous nephropathy.

PK samples will be collected as specified in Table 9.

On the days that PK samples are to be collected, study drug administration must occur under the supervision of the investigator (or his/her designee) after the predose PK sample is obtained. The actual timepoint that each sample is collected will be captured to the nearest minute in the eCRF. The time of study drug administration on Week 1 Visit and Week 4 Visit, and on the day before the Week 4 Visit, must be recorded in the eCRF. Further blood samples may be collected in the case of toxicity or adverse events induced by suspected drug-drug interaction and toxicity mechanisms, and such events must be recorded in the eCRF if they occur.

Blood samples (2 mL) for PK analysis will be collected into ethylenediaminetetraacetic acid collection tubes. Details concerning handling of the PK plasma samples, including labeling and shipping instructions, will be provided in the Laboratory Manual for this study. Samples will be shipped to the designated bioanalytical laboratory to determine the plasma zanubrutinib concentrations using a validated method.

### 4.7 Safety Assessments

### 4.7.1 Physical Examination and Vital Signs

A complete physical examination will be performed at screening. A focused physical examination, as well as vital signs (sitting blood pressure, heart rate, and body temperature) and weight will be performed at each study visit. Height (cm) will be measured at screening only.

A complete physical examination includes an assessment of various body systems per standard of care at the study site and as clinically indicated by symptoms.

### 4.7.2 Electrocardiograms

An electrocardiogram will be obtained per local practice and read locally to confirm eligibility. A 12-lead ECG will be performed in triplicate at screening, confirmation assessment, at Week 52, at the End-of-Treatment (EOT) Visit, and as clinically indicated (eg, abnormal and clinically significant per the investigator). The ECG recordings should be performed after the patient has been resting for at least 10 minutes.

When coinciding with blood draws at the same timepoint, ECG assessment should be performed before blood draws. Patients should rest in a semi-recumbent or supine position for ≥10 minutes before ECG collection.

### 4.7.3 Concomitant Medications

Any new medications, changes in ongoing medications or procedures, and medications discontinued within 30 days before run-in period and since the prior study visit will be recorded. Concomitant medications should be collected at screening and during the run-in period, during the treatment period, and during the extension observation period.

### 4.7.4 Adverse Events

Severity of adverse events will be assessed and recorded throughout the study.

Characterization of adverse events will include severity, duration, and time to onset.

All adverse events, including serious adverse events, will be collected.

### 4.8 Efficacy Assessments

### 4.8.1 Complete Remission

A complete remission is defined as:
UPCR (based on 24-hour urine collection)≤0.3, AND
A stable eGFR (remains unchanged or decreases by <15% compared with the baseline)

### 4.8.2 Partial Remission

A partial remission is defined as:
UPCR (based on 24-hour urine collection) >0.3 and ≤3.5, and >50% decrease compared with the baseline, AND
A stable eGFR (remains unchanged or decreases by <15% compared with the baseline)

### 4.8.3 Overall Remission

Patients with overall remission are those achieving either complete remission or partial remission.

### 4.8.4 Definitions

### 4.8.4.1 Relapse

A relapse is defined as reappearance of UPCR (based on 24-hour urine collection) >3.5 after complete or partial remission.

Note: If there is any potential cause accompanied with the recurrence of proteinuria, a confirmatory 24-hour urine protein test will be repeated within 2 weeks or after resolution of the potential cause.

### 4.8.4.2 Treatment Failure

A treatment failure is defined as meeting any of the following:
At Week 24, reduction in UPCR (based on 24-hour urine collection) is <25% compared with the baseline (confirmed with a repeated measurement within 2 weeks), UPCR (based on 24-hour urine collection) is >3.5, AND, for patients who had positive anti-PLA2R Abs at baseline, antibody level is >50 RU/mL
At Week 52, reduction in UPCR (based on 24-hour urine collection) is <50% compared with the baseline (confirmed with a repeated measurement within 2 weeks), UPCR (based on 24-hour urine collection) is >3.5, AND, for patients who had positive anti-PLA2R Abs at baseline, where immunological response is not achieved.
Initiation of immunosuppressive medication (including rescue therapy) other than the study drug
Diagnosis of ESKD or initiation of dialysis
Relapse by Week 104
Has not achieved complete remission or partial remission by Week 104
Death attributable to study treatment or primary disease

If a treatment failure occurs, the patient will be withdrawn from the study drug, and the patient's treatment will be adjusted according to clinical guidelines and local practice.

### 4.8.4.3 Immunological Response:

Immunological response is defined as anti-PLA2R antibody level reduced from baseline to less than 14 RU/ml.

Note: To quantify anti-PLA2R Ab levels in the patients, a commercial quantitative ELISA assay (EUROIMMUN AG) for the IgG-specific isotype of anti-PLA2R, according to manufacturer instructions.

### 4.8.4.4 Baseline

Baseline is defined as the last measurement before the first dose of the study drug.

### 4.9 Patient-Reported Outcomes

PROs will continue to be assessed until withdrawal of consent, death, the patient lost to follow-up, or the end of the study, whichever occurs first, regardless of study treatment discontinuation. Patients should complete the KDQoL-36, EQ-5D-5L, and PGI-S per the Schedule of Assessments (Table 3) before the study drug is administered and before performing any other procedures at the indicated visits for the 3 groups in Part 2.

KDQOL-36 is a validated PRO instrument, and it includes the 12-Item Short Form Survey (SF-12) as generic core scales (physical functioning: 2 items, role-physical: 2 items, pain: 1 item, general health: 1 item, emotional well-being: 2 items, role-emotional: 2 items, social functioning: 1 item, and energy/fatigue: 1 item) plus 3 additional kidney disease-related scales: burden of kidney disease (4 items), symptoms/problems of kidney disease (12 items), and effects of kidney disease (8 items) scales. This instrument is the most frequently used PRO instrument in the clinical settings as well as in clinical studies to measure health-related quality of life in patients with kidney diseases, and it has been linguistically validated in 50 languages. Higher scores indicate better HRQoL.

The EQ-5D-5L is one of the most frequently used PRO instruments in the clinical setting and in clinical studies measuring general HRQoL, and it is available in more than 100 linguistically validated languages. It consists of 5 domains (mobility, selfcare, usual activities, pain/discomfort, and anxiety/depression) and a visual analog scale (VAS). Higher scores indicate better HRQoL.

### 4.10 Laboratory Assessments

Samples for UPCR (based on 24-hour urine collection), urine creatinine and protein excretion, anti-PLA2R-antibody and CD4+ T cell count will be evaluated in a central lab. Other samples for protocol-specified hematology, chemistry, and coagulation profiles, etc, will be evaluated in a local laboratory unless otherwise specified.

A detailed description of the procedures for sample collection, handling, storage, and shipment of the laboratory samples and all materials such as test tubes and labels will be provided in the Laboratory Manual.

Laboratory assessments including but not limited to serum chemistry, hematology, coagulation, serum immunoglobulins, and urinalysis will be tested at the timepoints specified in the Schedule of Assessments (Table 2 and Table 3) and may also be performed as medically necessary. Certain elements of laboratory assessments will be collected.

### 4.10.1 Urinalysis, Urine Protein Creatinine Ratio, 24-Hour Urine Collection

Urinalysis and UPCR (based on 24-hour urine collection) are required to be tested at the scheduled visits specified in the Schedule of Assessments (Table 2 and Table 3); however, sample collection will not be performed in female patients during menstruation. Unscheduled collection of urine samples should be supplemented within 7 days after menstruation, and this should not be regarded as a protocol deviation. The 24-hour Urine Collection Instructions will be provided to the patients and must be followed to ensure that the urine collection is complete.

### 4.10.2 Hematology

Required hematology tests will be done at the scheduled visits specified in the Schedule of Assessments (Table 2 and Table 3). Hematology includes hemoglobin, hematocrit, platelet count, red blood cell count, and white blood cell count with differentials.

### 4.10.3 Chemistry

Required serum chemistry tests will be done at the scheduled visits specified in the Schedule of Assessments (Table 2 and Table 3). Serum chemistry includes sodium, potassium, chloride, glucose, blood urea nitrogen (or urea), creatinine, total calcium, phosphate/phosphorus, magnesium, total bilirubin, total protein, albumin, alanine aminotransferase, aspartate aminotransferase, lactate dehydrogenase, alkaline phosphatase, and creatine kinase.

The estimated glomerular filtration rate will be calculated from serum creatinine using the Chronic Kidney Disease Epidemiology Collaboration (CKD-EPI) equation.

### 4.10.4 HbAlc

Hemoglobin Alc (HbAlc) tests will be performed at the scheduled visits specified in the Schedule of Assessments (Table 2 and Table 3).

### 4.10.5 Trough Concentration of Tacrolimus

In Part 2, trough concentration of tacrolimus will be monitored at the scheduled visits specified in the Schedule of Assessments (Table 3).

### 4.10.6 Serum Immunoglobulins

Quantitative serum immunoglobulins (IgG, IgM, and IgA) will be measured at the timepoints specified in the Schedule of Assessments (Table 2 and Table 3).

### 4.10.7 Coagulation

The coagulation profile includes prothrombin time, international normalized ratio, and activated partial thromboplastin time. The coagulation profile will be performed at specified visits (Table 2 and Table 3).

### 4.10.8 Lipid Panel

The lipid panel includes total cholesterol, low-density lipoprotein (LDL), high-density lipoprotein (HDL), and triglycerides. Required serum lipid tests will be done at the scheduled visits specified in the Schedule of Assessments (Table 2 and Table 3).

### 4.10.9 Hepatitis B and C Testing

Hepatitis B and C serologic markers and/or viral load will be tested at screening. Hepatitis B testing may be performed by a local laboratory if the laboratory is able to perform the test to the required sensitivity (<20 IU/mL for HBV DNA); otherwise, the results must be confirmed by the central laboratory. The hepatitis B testing includes HBsAg, HBcAb, and hepatitis B surface antibody (HBsAb), as well as HBV DNA by PCR if the patient is negative for HBsAg but positive for HBcAb (regardless of HBsAb status). HCV antibody will be tested. Patients with positive HBsAg and/or detectable level of HBV DNA or positive HCV antibody are ineligible.

Patients who are HBcAb positive (HBsAg negative) and HBV DNA negative must undergo HBV DNA monitoring by PCR. These patients should receive prophylactic antiviral treatment in consultation with a local HBV expert. If a patient is being treated prophylactically with antiviral therapy, HBV DNA screening by PCR must be done at least every 90 days.

Table 4 describes how the results for HBV and HCV testing at screening relate to study eligibility.

TABLE 4
Active Hepatitis B or Hepatitis C Infection

| Screening | | |
|---|---|---|
| Assessment | Meeting Inclusion Criteria | To Be Excluded |
| HBV | HBsAg (−) and HBcAb (−) | HBsAg (+) |
| | HBsAg (−) and HBcAb (+) | HBsAg (−) and |
| | HBV DNA “Not detected” | HBcAb (+) |
| | Perform monitoring of HBV DNA | HBV DNA |
| | (at least every 90 days for | detected |
| | patients receiving | |
| | prophylactic antiviral therapy) | |
| HCV | Antibody (−) | Antibody (+) |

Abbreviations:
DNA, deoxyribonucleic acid;
HBcAb, hepatitis B core antibody;
HBsAg, hepatitis B surface antigen;
HBV, hepatitis B virus;
HCV, hepatitis C virus.

### 4.10.10Pregnancy Test

For women of childbearing potential, a laboratory-based highly sensitive serum pregnancy test must be performed at screening and be documented as negative. Any female patient who is pregnant will not be eligible for the study. A urine pregnancy test will be performed before administration of study drug at all the other visits (excluding screening) specified in the Schedule of Assessments (Table 2 and Table 3). A pregnancy test must be performed at the Safety Follow-up Visit for patients who prematurely discontinued the study drug. If a urine pregnancy test is positive, it must be confirmed by a serum pregnancy test. A patient who has a positive pregnancy test result at any time after the study drug administration will be immediately withdrawn from participation in the study.

### 4.10.11Renal Biopsy

Renal biopsy results should be used to determine the eligibility of patients. The renal biopsy results should include light, immunofluorescence, and electron microscopy image descriptions and confirm that the subepithelial deposits are seen on the electron microscopy. If no previous renal biopsy results are available, a renal biopsy must be performed before treatment assignment.

### 4.10.12COVID-19 Test

A COVID-19 test will be conducted to establish eligibility according to local practice.

### 4.11 Biomarkers

Peripheral blood samples and urine samples will be collected from patients at different timepoints specified in the Schedule of Assessments (Table 9) to explore the association of biomarkers with glomerular disease pathogenesis, disease activity, and the response to zanubrutinib. Such assessments will include protein biomarkers (eg, autoantibodies, cytokines, and proteomic signatures), gene expression signatures, primary membranous nephropathy-associated genetic polymorphisms, and/or other pharmacodynamics (PD) biomarkers such as BTK occupancy and BTK related immune cell activity.

Collection of blood samples for determining serum anti-PLA2R Ab level as measured by enzyme-linked immunosorbent assay by the central laboratory will be mandatory for all patients during screening, and for all patients with positive anti-PLA2R Abs at the end of the run-in period and at specified timepoints during the treatment period.

Collection of blood samples for determining serum anti-THSD7A Ab level as measured by enzyme-linked immunosorbent assay by the central laboratory will be performed for the patients with confirmed negative anti-PLA2R Ab at initial screening. Blood samples for anti-THSD7A Ab will be collected at confirmation assessment and then the following visit if the result is positive at baseline. Blood samples for BTK occupancy will be collected from 10 to 15 patients in Part 1, and 10 to 15 randomized patients each from Group A and Group B in Part 2.

Blood samples for BTK related immune cell activity analysis, cytokine analysis, protein signature analysis, gene expression signature analysis, primary membranous nephropathy-associated genetic polymorphism analysis, and urine samples will be collected from all randomized patients at different timepoints during treatment to explore the association of certain biomarkers with disease pathogenesis and patients' clinical response to zanubrutinib.

Shipping, storage, and handling of blood samples and urine samples for the assessment of biomarkers will be managed by a central laboratory. Samples will be individually processed and stored as described in the Laboratory Manual.

Archived coded samples may be used for additional medical and/or scientific research projects and any regulatory purposes that are outside of the current study purpose and objectives (but always in compliance with applicable law). These research projects may relate to the diseases that are the subject of this study, validation of new techniques/assays, and/or associated potential medicines. This may include research to help developing ways to detect, monitor, or treat the target disease of this study.

For samples collected in Mainland China, only the biomarkers specified above will be tested. For samples collected outside of Mainland China, biomarkers may include but are not limited to the above specified tests.

### 4.12 Unscheduled Visits

Unscheduled visits may be performed at any time as clinically indicated and may include vital signs/focused physical examination as clinically indicated, adverse event review, concomitant medications and procedures review, and hematology and chemistry laboratory assessments. The date and reason for the unscheduled visit must be recorded in the source documentation.

If an unscheduled visit is necessary to assess toxicity, diagnostic tests may be performed based on investigator assessment as appropriate, and the test results should be entered on the unscheduled visit page of the eCRF.

### 4.13 Missed Visit(s) During COVID-19 Epidemic

For visits that are missed due to COVID-19, any assessments required on that visit may be performed at an external local hospital rather than at the investigator site and recorded in the electronic data capture (EDC) system. Investigators should ensure that, at minimum, key safety assessments, as indicated in the protocol (see Section 4.7), are conducted. A telephone or video assessment by the investigator should include a review of any new adverse events or changes to any ongoing adverse events, as well as any changes to concomitant medications. Also, investigators should discuss patients' current study drug supply and review any questions patients may have regarding their study drug. The findings of the telephone/video discussion should be documented in the patients' chart as appropriate.

Regarding special missed visit(s) because of COVID-19:
The Week 1 Day 1 visit should not be missed
An unscheduled visit should be performed within 4 weeks, and samples for safety assessments, laboratory assessment, pharmacokinetics, and biomarkers on the missing visit should be collected at the unscheduled visit (if the next visit is within 4 weeks, the unscheduled visit could be skipped)
For patients with study drug administration interruption because of COVID-19, PK sampling should be skipped on that visit.

### 4.14 Treatment Period

The treatment period will start from the date of the assignment of study treatment and continue until the last dose of study drug. The date of the first dose should be within one day after treatment assignment.

Patients may discontinue the study drug for any of the following reasons:
Adverse event(s)
Consent withdrawal by the patient
Investigator's decision
Lost to follow-up
Treatment failure
Other reasons (including positive pregnancy test)

Patients may voluntarily withdraw the consent to receive treatment at any time.

### 4.15 Safety Follow-Up

A patient who prematurely discontinues the study drug and is unwilling to complete subsequent clinic visits will be asked to return for a Safety Follow-up Visit approximately 30 days after the last dose of the study drug to collect treatment-emergent adverse events (for the definition, refer to Section 7.4.2), including treatment-emergent adverse events that may have occurred or have been ongoing after the patient has discontinued the study drug. A laboratory assessment will only be required if the patient has an ongoing laboratory abnormality at the previous visit which is considered by the investigator to be related to study drug. If the patient is unable to return to the clinic and no laboratory assessment is necessary, the investigator or his/her designee will contact the patient or guardian to collect this information. Refer to the Schedule of Assessments (Table 2 and Table 3) for the assessments to be performed at the Safety Follow-up Visit. 4.16 Premature Discontinuation From the Study Drug Discontinuation from the study drug does not necessarily mean discontinuation from follow-up or termination of study participation. Patients who have discontinued from the study drug should complete the EOT Visit (within 1 week after the last dose) as early as possible to support the final efficacy and safety analyses. The reason for premature discontinuation from the study drug will be documented in the source documents and the eCRF.

Compliant patients who discontinue the study drug will be encouraged to return for all scheduled clinic visits till the planned Week 104 Visit. During visits after study drug discontinuation, procedures and assessments listed below should be completed at minimum:
Vital signs
24-hour urine collection at the EOT Visit
eGFR
Adverse events reported until 30 days after the last dose

If a patient is unwilling to complete all scheduled clinic visits, he/she should complete the EOT Visit, the Safety Follow-up Visit 30 days after the last dose of the study drug, the planned Week 52 Visit, Week 76 Visit, and Week 104 Visit. If a patient is unwilling to return for any study visits, at a minimum, UPCR (based on 24-hour urine collection) and blood creatinine should be assessed at the EOT visit.

### 4.17 End of Study

For patients who have completed the Week 104 visit, their study participation will be ended. For patients who prematurely discontinue the study drug, attempts should be made to follow them until the planned Week 104 Visit. Their participation in the study will be ended after their Safety Follow-up Visit or the planned Week 104 Visit, whichever comes later.

Complete withdrawal from the study (including treatment and all follow-up visits) will occur under the following circumstances:
Consent withdrawal by the patient
Study termination by the sponsor
Lost to follow-up
Any other circumstances at the investigator's discretion
Death
Other

Patients may voluntarily withdraw consent from the study at any time. Patients requesting withdrawal should be informed by the investigator that withdrawal of consent for follow-up will jeopardize the public health value of the study. The investigator should explicitly ask patients who withdraw about the contribution of possible adverse events to their decision to withdraw consent, and any adverse event information elicited should be documented. Preferably, the patient should withdraw consent in writing and, if the patient or the patient's representative refuses or is physically unavailable, the site should document and sign the reason for the patient's failure to withdraw consent in writing.

### 4.18 Lost to Follow-up

Every reasonable effort should be made to contact any patient apparently lost to follow-up during the study to complete study-related assessments, record outstanding data, and retrieve the study drug.

More specifically, an attempt to contact the patient by phone should be made; however, if phone contact is unsuccessful, the patient or alternative contacts (eg, primary care providers, referring physicians, and relatives) can be contacted by mail. Such efforts should be documented in the patient's source documents.

If all efforts fail to establish contact, the patient will be noted as lost to follow-up.

### Study Treatment

### 5.1 Study Treatment Preparation and Dispensation

### 5.1.1 Packaging and Labeling

Zanubrutinib capsules will be provided in child-resistant high-density polyethylene (HDPE) bottles with an induction seal and bottle label.

Refer to the pharmacy manual for specifics on packaging and label content.

Tacrolimus oral capsules will be sourced from commercial supplies. The labels will include any additional regionally specific clinical requirements as appropriate. Refer to the Pharmacy Manual for specifics on packaging and label content.

The contents of the label will be in accordance with all applicable local regulatory requirements.

### 5.2 Dosage and Administration

### 5.2.1 Zanubrutinib

Zanubrutinib will be dispensed by the study center personnel to patients at scheduled study visits to ensure an adequate drug supply for administration at home throughout the treatment phase as detailed in the Pharmacy Manual. The investigator is to instruct the patient to take the study drug exactly as prescribed and at the same time each day of dosing. Patients will be requested to bring their unused medication, and all empty bottle packs, to the center at each visit. All dosages prescribed and dispensed to the patient and all dose changes including reason for dose changes during the study must be recorded on the appropriate eCRF.

For Part 1 and Part 2 Group A, zanubrutinib will be administered as two 80-mg capsules by mouth twice a day (160 mg twice a day) with or without food. For Part 2 Group B, zanubrutinib will be administered as two 80-mg capsules by mouth once a day (160 mg once a day) with or without food. Patients will take zanubrutinib capsules with water at approximately the same time every day, with a minimum of 8 hours between consecutive doses. Zanubrutinib capsules should not be opened, broken, or chewed at any time.

Patients should be instructed that if a dose of the study drug is not taken at the scheduled time, they should skip the study drug if the time to next dose is less than 8 hours and return to normal dosing with next dose. If a patient vomits after taking the zanubrutinib capsules, that dose should not be repeated.

On the days of PK blood sampling, study drug administration for patients will occur at the study site, after the predose blood sampling has occurred, and under the supervision of the investigator or his/her designee. The investigator or his/her designee must instruct the patient not to self-administer the study drug before the office visit on those days.

### 5.2.2 Tacrolimus

Patients in Group C will receive tacrolimus capsules. Tacrolimus oral capsules, with an initial dose of 0.05 mg/kg/day, divided into 2 doses, will be administered every 12 hours; adjusted to maintain a target trough blood concentration of 3 to 8 ng/mL for 52 weeks, then will be tapered off from Week 53, and finally should be discontinued by Week 64.

Tacrolimus should be administered consistently with or without food. If the patients consistently take tacrolimus with food, dietary guidance from the investigator should be followed. Predose blood concentration (trough concentration) of tacrolimus should be monitored every 2 weeks during the first 2 months of the treatment period and as specified in Schedule of Assessment (Table 3) after that. If the target blood level has not been achieved, an unscheduled visit would be arranged after dose adjustment (refer to Section 5.5.3) before Week 53.

### 5.3 Overdose

Dosage of tacrolimus will be adjusted according to the blood drug concentration and the tolerability. Any dose of zanubrutinib in excess of that specified in this protocol is considered to be an overdose. Adverse events associated with an overdose or incorrect administration of study drug will be recorded on the adverse event eCRF. Any serious adverse events associated with an overdose or incorrect administration are required to be reported within 24 hours of awareness via the serious adverse event reporting process. There are no specific antidotes for zanubrutinib or tacrolimus. In an event of an overdose, patients should be closely monitored and given appropriate supportive treatment.

### 5.4 Precautions

Grapefruit or grapefruit juice should not be consumed in Group C and should be given with caution in Group A and Group B.

For detailed information on warnings and precautions for tacrolimus, refer to the prescribing information and Summary of Product Characteristics for the agents.

### 5.4.1 Surgery and Procedures

Susceptibility to bleeding has been observed with BTK inhibitors. Study treatment with zanubrutinib should be held for 3 to 7 days before and after surgery, depending upon the type of surgery and the risk of bleeding.

### 5.5 Dose Modification

### 5.5.1 Dose Modification for Zanubrutinib for Hematologic Toxicity

Every effort should be made to administer the zanubrutinib according to the planned dose and schedule. Dosing will be held for individual patients under any of the following conditions, based on the investigator's assessment:
Neutrophil count decreased <1.0×10⁹/L (<1000/mm³);
Platelet count decreased <25.0×10⁹/L (<25,000/mm³);
Platelet count decreased <50.0×10⁹/L (<50,000/mm³) associated with significant bleeding

For the first occurrence of hematologic toxicity, treatment may restart upon recovery of the toxicity to mild or baseline.

If the same event reoccurs, each dose will be reduced by half until recovery of the toxicity to mild or baseline. If the same event reoccurs for the third time, zanubrutinib should be discontinued. For patients with thrombocytopenia (platelet count decreased <50.0×10⁹/L [<50,000/mm³]) associated with significant bleeding requiring medical intervention, the dosage will be discussed with the medical monitor.

### 5.5.2 Dose Modification for Zanubrutinib for Nonhematologic Toxicity

Zanubrutinib should be held for any severe bleeding and should be permanently discontinued for any severe hemorrhage related to zanubrutinib, with the exception of those where the underlying condition can be fully treated (eg, gastric ulcer resulting in gastrointestinal bleeding) and the risk of rebleeding is deemed acceptable. Zanubrutinib should be permanently discontinued for any grade of intracranial hemorrhage (Table).

For severe nonhematological toxicities suspected to be related to zanubrutinib, except for hypertension adequately controlled with oral medication or asymptomatic laboratory events (laboratory events indicating liver or renal dysfunction will not be considered asymptomatic laboratory events), zanubrutinib will be held until recovery to mild or baseline, and then restarted at the original dose level. If the event recurs at severe intensity, zanubrutinib will be held until recovery to mild or baseline and restarted at half of each dose. If the severe event recurs again, zanubrutinib will be permanently discontinued. For patients experiencing atrial fibrillation that is symptomatic and/or incompletely controlled, after the atrial fibrillation is adequately controlled, zanubrutinib may be restarted at either the original dose or half of the dose, at the investigator's discretion (Table).

For information on the suspension of zanubrutinib based on the results of hepatitis B or hepatitis C testing, see Section 4.10.9.

For any active infection or significant exposure to any infection, the investigator should consider whether to interrupt administration of zanubrutinib. Similarly, if a patient presents with signs or symptoms where severe opportunistic infection is considered, zanubrutinib should be interrupted.

TABLE 5
Dose Modification Steps for Nonhematologic Toxicity

| Toxicity | Actions taken for zanubrutinib | Restart dose |
|---|---|---|
| Severe bleeding not | Hold until recovery to mild. | Restart at either the |
| considered related | | original dose or administer |
| to study drug | | half of each dose, at the |
| | | treating investigator's |
| | | discretion. |
| Severe bleeding | Hold until bleeding has fully resolved. | Administer half of each |
| considered related | If an underlying condition cannot be | dose. |
| to study drug | treated to full resolution, permanently | |
| | discontinue the study drug. | |
| Any grade intracranial | Permanently discontinue the study | Not applicable. |
| hemorrhage | drug. | |
| Atrial fibrillation that is | Hold until atrial fibrillation is | Restart at either the |
| symptomatic and/or | controlled. | original dose or administer |
| incompletely controlled | | half of each dose, at the |
| | | treating investigator's |
| | | discretion. |
| Other severe toxicities | Hold until recovery to less than or | Restart at the original |
| considered related to | equal to baseline. | dose. |
| study drug, including | | |
| inadequately controlled | | |
| hypertension and/or liver | | |
| or renal laboratory value | | |
| abnormalities | | |
| Severe opportunistic | Hold until the investigator | Restart at either the |
| infections; active | confirms the symptoms and signs | original dose or at a half |
| infection or significant | of the infection have resolved. | dose, at the treating |
| exposure to any infection | | investigator's discretion. |

### 5.5.3 Dose Interruptions and Modifications for Tacrolimus

The daily tacrolimus dosage should be adjusted in any of the following conditions:
Blood trough concentration of tacrolimus is out of the target range (3 to 8 ng/mL).
≥30% increase in serum creatinine during the first 24 weeks of tacrolimus treatment.
Any controllable adverse events under the treatment of tacrolimus at the discretion of investigators.

In patients with a ≥30% increase in serum creatinine during the first 24 weeks of tacrolimus treatment, the dose of tacrolimus should be tapered by ≥30% when potential underlying conditions such as excessive diuretic therapy or non-renal volume depletion have been ruled out. If azotemia does not resolve ≤2 weeks after dose modification, tacrolimus should be discontinued and patient can be treated according to clinical guidelines and local practice. Patients needing such changes will be considered as having had treatment failure. After treatment failures, visits specified by the protocol should be made whenever possible.

Tacrolimus should be interrupted at the discretion of investigators when a severe adverse event occurs. Tacrolimus could be restarted upon resolution of adverse event other than intolerant nephrotoxicity.

### 5.5.4 Discontinuation of Study Treatment due to Dose Interruption

For any patients with interruption of study drug (zanubrutinib or tacrolimus) for >, 8 weeks, whether to discontinue the study drug should be discussed with the medical monitor.

### 6 Prior and Concomitant Therapy

### 6.1 Prior Therapy

Prior use of any of the following therapies is prohibited:
Prior exposure to a BTK inhibitor
Receipt of any prohibited drug defined in Table and Table before the assignment of study treatment
Receipt any of the following:
   a. Any live vaccine within 4 weeks before the assignment of study treatment
   b. Bacillus Calmette-Guerin (BCG) vaccine within 1 year before the assignment of study treatment
   c. Any prohibited medications listed in Section 6.2.3 if the washout period is not met
   d. Prior hematopoietic stem cell transplantation
   e. Plasmapheresis or leukapheresis within 12 weeks before the assignment of study treatment

TABLE 6
Drugs Prohibited Before the Assignment of Study Treatment

| | Duration before the |
|---|---|
| | assignment of study |
| Drugs prohibited | treatment |
| Any investigational product | <4 weeks or |
| (small molecule or biologic agent) | 5 half-lives |
| | of the product |
| Systemic prednisone | <4 weeks |
| (or other systemic glucocorticoids) | |
| Any other immunosuppressive agents, | |
| eg, mycophenolate mofetil and Tripterygium | |
| Leflunomide | <12 weeks |
| (unless an adequate cholestyramine wash- | |
| out has been done for leflunomide) | |
| Intravenous or oral cyclophosphamide, | <6 months |
| calcineurin inhibitors | |
| (including but not limited to tacrolimus, | |
| cyclosporine, and voclosporine) | |
| B cell-targeting or plasma cell-targeting therapy | <9 months |
| (including but not limited to | |
| anti-CD20 mAb, anti-CD19 mAb, | |
| anti-CD38 mAb, anti-BAFF, and | |
| anti-BAFF/APRIL mAb) | |

TABLE 7
Prohibited Prior And Concomitant Therapies

| | | | |
|---|---|---|---|
| Anti-TNFα | infliximab | Anti-IL17 mAb | secukinumab |
| agents | etanercept | | ixekizumab |
| | adalimumab | | bimekizumab |
| | golimumab | Anti-IL12/23 p40 mAb | ustekinumab |
| | certolizumab pegol | Anti-IL23p19 mAb | guselkumab |
| | | | risankizumab |
| Anti-CD20 | rituximab | Anti-IL1 agents | anakinra |
| agents | obinutuzumab | Anti-CD19 mAb | inebilizumab |
| | ocrelizumab | Anti-CD38 mAb | daratumumab |
| Anti-BAFF | belimumab | | felzartamab |
| mAb | | | |
| TACI-Ig | atacicept | azathioprine | |
| | telitacicept | cyclophosphamide | |
| Anti-IL6 mAb | tocilizumab | voclosporine | |
| | siltuximab | sirolimus | |
| CTLA4 Ig | abatacept | cyclosporine | |
| Approved BTK | ibrutinib | leflunomide | |
| inhibitors | acalabrutinib | tripterygium | |
| (China/the | orelabrutinib | | |
| United States | | | |

Abbreviations:
BAFF, B-cell activation factor;
BTK, Bruton tyrosine kinase;
CTLA, cytotoxic T lymphocyte-associated antigen;
Ig, immunoglobulin;
IL, interleukin;
mAb, monoclonal antibodies;
TACI, transmembrane activator and calmodulin ligand interactor;
TNF, tumor necrosis factor.

### 6.2 Concomitant Therapy

All concomitant medications taken during the study will be recorded in the eCRF with indications and dates of administration.

Prophylactic measures, for the prevention of bacterial, fungal, or viral infections should be started before or concomitant with study treatment per institutional standards.

For special infections, such as latent tuberculosis infection or HBV reactivation, refer to the following:
Patients with latent tuberculosis or who have had recent close contact with a person with active tuberculosis should complete the appropriate treatment (refer to Section 3.2) before randomization.
Patients with HBsAg (−), HBcAb (+), and undetectable HBV DNA (<20 IU/mL) should start prophylactic antivirus treatment (entecavir or tenofovir alafenamide fumarate) before or concomitant with study treatment. HBsAg, alanine aminotransferase, and HBV DNA should be monitored during the entire study period.

### 6.2.1 Optimal Supportive Care for Primary Membranous Nephropathy

All patients will receive optimal supportive care during the run-in period and the entire study unless contraindicated or otherwise noted. The maximally tolerated or allowed dose of ACEI or ARB should be given during the run-in period. ACEI or ARB will not be discontinued when there is a modest and stable increase of blood creatinine ≤30%; ACEI or ARB combination or combination with a renin inhibitor is prohibited. The dose of ACEI or ARB must remain stable throughout the study period (stable therapy defined as <25% dose increase).

For the patients who skip the run-in period (with adequate blood pressure control [blood pressure <130/80 mmHg, measured on ≥2 occasions in the month before randomization] and eGFR<60 mL/min/1.73 m² and/or urine protein >8 g/24 hours at initial screening), ACEI or ARB will be started within 12 weeks after randomization and remain stable after achieving the maximally tolerated or allowed dose during the entire study period.

Blood pressure should be well controlled at the end of run-in period and be confirmed as <130/80 mmHg, measured on ≥2 occasions in the last month before randomization. If the blood pressure control target cannot be achieved with ACEI or ARB, additional antihypertension medications are recommended in the following order: a loop diuretic, a cardio-selective β-blocker, a non-dihydropyridine calcium channel blocker, and clonidine.

At the start of the run-in period, statins will be started as a first-line treatment for patients with nephrotic syndrome accompanied with hyperlipidemia. Treatment of hyperlipidemia in patients with nephrotic syndrome may follow the guidelines that apply to the general population.

Anti-hyperlipidemia treatment should be started ≥2 weeks before the assignment of study treatment.

### 6.2.2 Permitted Medications

Anticoagulation treatment: For patients with thromboembolic events such as venous thrombosis, arterial thrombosis, pulmonary embolus, and nonvalvular atrial fibrillation, full dose anticoagulation is required for 6 to 12 months for the duration of nephrotic syndrome.

Prophylactic anticoagulation is recommended when serum albumin is less than 20 g/L accompanied with any of the following: urine protein >10 g/24 hours, body mass index >35 kg/m², or prolonged immobilization.

Use of anticoagulants and the dosage regimen should be determined according to clinical guidelines, the clinical decision of investigator, the specific clinical characteristics of the patient, and should consider the risk of thromboembolism and anticoagulant-induced serious bleeding events. Nonsteroidal anti-inflammatory drugs should be avoided unless for prophylaxis of cardiovascular, cerebrovascular, or thrombotic events.

CYP3A inhibitors in Group C: some CYP3A inhibitors which may require dose reduction of tacrolimus, such as diltiazem or schisandra, are permitted concomitant medications combined with tacrolimus at the discretion of the investigator. Trough concentrations of tacrolimus should be monitored and maintained within the target range.

### 6.2.3 Prohibited Medications

Concomitant use of any of the following medications is prohibited while the patient is receiving the study drug; if any of them is used, the study drug will be discontinued:
Any other investigational products (small molecule or biologic agent)
Any other BTK inhibitors
Any systemic use of immunosuppressants (including glucocorticoids, refer to Table), except rescue therapy.
ACEI and ARB combination or ACEI/ARB in combination with a renin inhibitor
Any live vaccine during the study or within 3 months after the last dose of the study drug
Strong CYP3A4 inhibitors or inducers for the patients in Part 1 and Groups A and B in Part 2 (refer to Table).
Any drugs prohibited to be concurrently used with tacrolimus in Group C in Part 2, such as grapefruit or grapefruit juice, and cyclosporine.

TABLE 8
CYP3A Inhibitors And Inducers
Strong CYP3A Inhibitors

| | |
|---|---|
| Antibiotics: | clarithromycin, telithromycin, troleandomycin |
| Antifungals: | itraconazole, ketoconazole, posaconazole, voriconazole |
| Antivirals: | boceprevir, telaprevir |
| Other: | cobicistat, conivaptan, elvitegravir, mibefradil, nefazodone |

Strong CYP3A Inhibitors

| | |
|---|---|
| Protease | indinavir, lopinavir, nelfinavir, ritonavir, saquinavir, |
| inhibitors: | tipranavir |

Moderate CYP3A Inhibitors
CYP3A4, CYP3A5, CYP3A7

| | |
|---|---|
| Antibiotics: | ciprofloxacin, erythromycin |
| Antifungals: | fluconazole, clotrimazole |
| Protease | amprenavir, atazanavir, darunavir/ritonavir, fosamprenavir |
| inhibitors: | |
| Calcium | diltiazem, verapamil |
| channel | |
| blockers: | |
| Tyrosine | |
| kinase | imatinib, crizotinib |
| inhibitors | |
| (anticancer): | |
| Food | grapefruit juice (Citrus paradisi juice) |
| products: | |
| Herbal | Schisandra sphenanthera |
| medications: | |
| Others: | amiodarone, aprepitant, casopitant, cimetidine, |
| | cyclosporine, dronedarone, tofisopam |

Strong/Moderate CYP3A Inducers
Avasimibe, carbazepine, mitotane, phenobarbital, phenytoin, rifabutin,
rifampin (rifampicin), St. John’s wort (Hypericum perforatum),
enzalutamide, mitotane, bosentan, efavirenz, etravirine, modafinil
Abbreviation: CYP3A, cytochrome P450, family 3, subfamily A.
Source: Food and Drug Administration Drug Development and Drug Interactions: Table of Substrates, Drug Development and Drug Interactions and Inducers.
Note:
The list of drugs in this table is not exhaustive. Please refer to the prescribing information and Summary of Product Characteristics to check for CYP3A inhibition or induction risks or contact the medical monitor of the protocol.

For a more complete list, please refer to http://medicine.iupui.edu/clinpharm/ddis/main-table/or to: Flockhart DA. Drug Interactions: Cytochrome P450 Drug Interaction Table. Indiana University School of Medicine. http://medicine.iupui.edu/flockhart/table.htm.

### 6.3 Potential Interactions Between the Study Drug and Concomitant Medications

### 6.3.1 Cytochrome P450-Inhibiting/Inducing Drugs and Zanubrutinib

Strong CYP3A inhibitors or CYP3A inducers are prohibited. Administration of zanubrutinib with moderate CYP3A inhibitors or CYP3A inducers (refer to Table) for a list of these medications) and grapefruit juice or Seville oranges should be done with caution, as they may affect the metabolism of zanubrutinib. If possible, patients are encouraged not to use moderate CYP3A inhibitors and inducers and to consider using alternative agents. The medical monitor should be consulted in these situations. Please refer to http://medicine.iupui.edu/clinpharm/ddis/main-table/ for a more complete list.

A clinical drug-drug interaction study (Study BGB-3111-108) indicated that zanubrutinib was a mild inducer of CYP3A4 and CYP2C19. Narrow therapeutic index drugs that are metabolized by CYP3A4 (alfentanil, cyclosporine, dihydroergotamine, ergotamine, fentanyl, pimozide, quinidine, and sirolimus) and CYP2C19 (eg, S-mephenytoin) should be used with caution, as zanubrutinib may decrease the plasma exposures of these drugs.

6.3.2 Drugs Potentially Interacting with Tacrolimus

For guidance regarding potential interactions with tacrolimus, investigators should consult the relevant prescribing information.

### 7 Statistical Methods and Sample Size Determination

The statistical analyses will be performed by the sponsor or designee after the data collection is completed and the database is locked and released. Details of the statistical analyses will be included in a separate Statistical Analysis Plan.

### 7.1 Study Endpoints

### 7.1.1 Primary Endpoint

### Part 1:

The primary endpoint is reduction in UPCR at Week 24 from baseline.

### Part 2:

The primary endpoint is complete remission status (Yes/No) (for definition of complete remission, refer to Section 4.8.1) at Week 104.

### 7.1.2 Secondary Endpoints

### Part 1:

The secondary endpoints include the following:
Treatment failure status (Yes/No) by Week 24
Immunological response status (Yes/No) at Week 24
Complete remission status (Yes/No) at Week 24, Week 52, Week 76, and Week 104
Overall remission status (Yes/No) at Week 24, Week 52, Week 76, and Week 104
Relapse status (Yes/No) by Week 104
Incidence and severity of treatment-emergent adverse events

### Part 2:

The key secondary endpoint is overall remission status (Yes/No) (for definition of overall remission, refer to Section 4.8.3) at Week 104.

The secondary endpoints also include the following:
Complete remission status (Yes/No) at Week 24, Week 52, and Week 76
Overall remission status (Yes/No) at Week 24, Week 52, and Week 76
Treatment failure status (Yes/No) by Week 24, Week 52, Week 76, and Week 104
Time to first complete remission: the time from the date of randomization to the date of the first complete remission
Time to first overall remission: the time from the date of randomization to the date of the first overall remission
Relapse status (Yes/No) by Week 104
Time to first relapse: the time from the date of first complete or partial remission to the date of the first relapse
HRQoL as measured using the KDQoL-36 and EQ-5D-5L
≥30% eGFR reduction from baseline to Week 52 and Week 104 (Yes/No)
Incidence and severity of treatment-emergent adverse events

### 7.1.3 Exploratory Endpoints

### Part 1:

The exploratory endpoints include the following:
Pharmacokinetics of zanubrutinib in patients with primary membranous nephropathy
Potential biomarkers associated with the efficacy of zanubrutinib in peripheral blood and/or urine samples, including but not limited to autoantibodies, cytokines, gene expression signatures, primary membranous nephropathy-associated genetic polymorphism, and/or other PD biomarkers, such as BTK occupancy and BTK-related immune cell activity

### Part 2:

The exploratory endpoints include the following:
Pharmacokinetics of zanubrutinib in patients with primary membranous nephropathy
Immunological response status (Yes/No) at Week 12, Week 24, Week 52, and Week 104 in patients with positive anti-PLA2R Abs at baseline
Change in serum anti-PLA2R Ab titers from baseline to Week 104
Time to immunological response: the time from the date of randomization to the date of immunological response
Complete remission status (Yes/No) at Week 104 based on baseline status of anti-PLA2R Abs
Overall remission status (Yes/No) at Week 104 based on baseline status of anti-PLA2R Abs
Potential biomarkers associated with the efficacy of zanubrutinib in peripheral blood and/or urine samples, including but not limited to autoantibodies, cytokines, gene expression signatures, primary membranous nephropathy-associated genetic polymorphism, and/or other PD biomarkers, such as BTK occupancy and BTK-related immune cell activity
Severity of patient-reported symptoms as measured by PGI-S

### 7.2 Statistical Analysis

### 7.2.1 Randomization Methods

As discussed in Section 4.4.5, patients in Part 2 will be randomized by permuted block stratified randomization using the IRT system for this study.

### 7.2.2 Analysis Sets

The Full Analysis Set includes all randomized patients. Following the intent-to-treat principle, patients will be analyzed according to the treatment assigned at randomization. This will be the primary analysis set for efficacy analyses of Part 2.

The Safety Analysis Set includes all patients who have received >1 dose of the study drug. Patients will be analyzed according to their actual treatment received. For Part 1, the Safety Analysis Set will be used for all efficacy and safety analyses; for Part 2, the Safety Analysis Set will be used for all safety analyses.

The PK Analysis Set includes all patients who have received >1 dose of the study drug per the protocol and for whom any postdose PK data are available.

### 7.2.3 Patient Disposition

The number of patients treated, discontinued from the study drug and/or study, and those with important protocol deviations will be counted for both Part 1 and Part 2. The number of patients randomized will also be counted for Part 2. The primary reason for study drug and/or study discontinuation will be summarized according to the categories in the eCRF.

Important protocol deviations will be summarized and listed by each category.

### 7.2.4 Demographics and Other Baseline Characteristics

Demographic and other baseline characteristics will be summarized using descriptive statistics for both Part 1 and Part 2. Continuous variables include age, serum anti-PLA2R Ab titer, blood creatinine, body mass index, eGFR, serum albumin, serum cholesterol, systolic and diastolic blood pressure, time since initial membranous nephropathy diagnosis, weight, and UPCR (based on 24-hour urine collection). Categorical variables include country, sex, geographical region, and race. Categorical variables including positive serum anti-PLA2R Ab and stratification factors will also be summarized for Part 2.

### 7.2.5 Prior and Concomitant Therapy

Concomitant medications will be coded using the World Health Organization (WHO) Drug Dictionary drug codes. Concomitant medications will be further coded to the appropriate Anatomical Therapeutic Chemical (ATC) code indicating therapeutic classification. Prior and concomitant medications will be summarized and listed by drug and drug class. Prior medications will be defined as medications that stopped before the first dose of study drug. Concomitant medications will be defined as medications that 1) started before the first dose of study drug and were continuing at the time of the first dose of study drug, or 2) started on or after the date of the first dose of study drug up to 30 days after the patient's last dose.

### 7.3 Efficacy Analysis

### 7.3.1 Primary Efficacy Analysis for Part 1

Mean reduction in UPCR at Week 24 from baseline will be summarized in the Safety Analysis Set.

### 7.3.2 Primary Efficacy Analysis for Part 2

### 7.3.2.1 Primary Estimand

The primary estimand is defined in Section 1.2.

### 7.3.2.2 Primary Analysis for Primary Estimand

The statistical null hypotheses to be tested for the primary endpoint are as follows:

H₀₋₁: zanubrutinib 160 mg twice a day group (Group A) is not superior to the tacrolimus group (Group C) with respect to complete remission rate at Week 104.

H₀₋₂: zanubrutinib 160 mg once a day group (Group B) is not superior to Group C with respect to complete remission rate at Week 104.

Complete remission rate at Week 104 in the Full Analysis Set will be analyzed using a logistic regression model with treatment group and actual stratification factors as covariates. The odds ratio, with 95% confidence intervals and p-values, will be computed for comparisons of Group A versus Group C and Group B versus Group C utilizing the logistic regression model fitted.

### 7.3.2.3 Handling of Missing Values Not Related to Intercurrent Event

If a patient does not have the required data to compute a complete remission at Week 104, the missing data will be imputed as the value collected at Week 90. If the imputed value is still missing, the patient will be considered as a non-responder for complete remission at Week 104.

### 7.3.2.4 Sensitivity Analyses for Primary Endpoint/Estimand

Sensitivity analyses will be defined in the Statistical Analysis Plan as needed.

### 7.3.2.5 Supplementary Analyses

Supplementary analysis will be defined in the Statistical Analysis Plan as needed.

### 7.3.3 Secondary Efficacy Analyses for Part 1

Treatment failure status (Yes/No) by Week 24
Immunological response status (Yes/No) at Week 24
Complete remission status (Yes/No) at Week 24, Week 52, Week 76, and Week 104
Overall remission status (Yes/No) at Week 24, Week 52, Week 76, and Week 104
Relapse status (Yes/No) by Week 104

For the above secondary endpoints, the analyses will be similar to primary analysis.

### 7.3.4 Secondary Efficacy Analyses for Part 2

Overall remission status (Yes/No) at Week 104
Complete remission status (Yes/No) at Week 24, Week 52 and Week 76
Overall remission status (Yes/No) at Week 24, Week 52 and Week 76

For the above secondary endpoints, the analyses will be similar to primary analysis.

Treatment failure status (Yes/No) by Week 24, Week 52, Week 76, and Week 104

The number (percentage) of patients who had treatment failure by Week 24, Week 52, Week 76, and Week 104 will be summarized by treatment groups.

Time to first complete remission: the time from the date of randomization to the date of the first complete remission

Time to first complete remission will be analyzed using the Kaplan-Meier method. Kaplan-Meier survival probabilities for each treatment group will be plotted over time. A hazard ratio with 95% confidence interval will be computed to compare Group A versus Group C and Group B versus Group C using a Cox proportional hazard model adjusted for stratification factors.

Time to first overall remission: the time from the date of randomization to the date of the first overall remission

The analysis will be similar to the analysis of time to first complete remission.

Relapse status (Yes/No) by Week 104

The number (percentage) of patients with relapse by Week 104 will be summarized by treatment groups.

Time to first relapse: the time from the date of first complete or partial remission to the date of the first relapse

The analysis will be similar to the analysis of time to first complete remission.

HRQoL as measured using the KDQoL-36 and EQ-5D-5L

Descriptive statistics for all the PROs and their scales will be presented for all visits by treatment group. Compliance rate will also be summarized for each visit by treatment group.

A Mixed Model Repeated Measures (MMRM) analysis will be used to compare the mean change in symptoms, burden of kidney disease, and effects of kidney disease measured by KDQoL-36 from baseline to Week 24 and Week 52. This model will include treatment group, actual stratification factors, and visit as fixed effects; baseline score as a continuous covariate; treatment group by visit and baseline score by visit as interaction terms; and patient as a random effect. The differences between Group A versus Group C and Group B versus Group C will be presented with a 95% confidence interval and p values.

≥30% eGFR Reduction from Baseline to Week 52 and Week 104 (Yes/No)

The analyses of the proportion of patients with ≥30% eGFR reduction from baseline to Week 52 and Week 104 will be similar to primary analysis.

### 7.3.5 Exploratory Efficacy Analysis for Part 2

Immunological Response Status (Yes/No) at Week 12, Week 24, Week 52, and Week 104 in Patients with Positive Anti-PLA2R Abs at Baseline

The number (percentage) of patients achieving immunological response at Week 12, Week 24, Week 52, and Week 104 will be summarized by treatment group in patients with positive anti-PLA2R Abs at baseline.

Change in Serum Anti-PLA2R Ab Titers from Baseline to Week 104

Mean change in serum anti-PLA2R Ab titers from baseline to Week 104 will be summarized by treatment group.

Time to Immunological Response: The Time from the Date of Randomization to the Date of Immunological Response

Time to immunological response will be analyzed using Kaplan-Meier method. Kaplan-Meier survival probabilities for each treatment group will be plotted over time.

### Complete Remission Status (Yes/No) at Week 104 Based on Baseline Status of Anti-PLA2R Abs

The number (percentage) of patients achieving complete remission at Week 104 will be summarized by treatment group and baseline status of anti-PLA2R Abs.

### Overall Remission Status (Yes/No) at Week 104 Based on Baseline Status of Anti-PLA2R Abs

The number (percentage) of patients achieving overall remission at Week 104 will be summarized by treatment group and baseline status of anti-PLA2R Abs.

### Severity of Patient-Reported Symptoms as Measured by PGI-S

Descriptive statistics for PGI-S score will be presented for all visits by treatment group. Compliance rate will also be summarized for each visit by treatment group.

### 7.4 Safety Analyses

Safety will be assessed by monitoring and recording all adverse events. Laboratory values (chemistry, hematology, coagulation, or urinalysis), vital signs, physical examinations, and ECG findings will also be used in assessing safety. Descriptive statistics will be used to analyze all safety data by actual treatment group in the Safety Analysis Set for both Part 1 and Part 2.

### 7.4.1 Extent of Exposure

The extent of study drug exposure will be summarized descriptively as the duration of exposure (days), cumulative total dose received per patient (mg), dose intensity (mg/day), and relative dose intensity (%).

The number (and percentage) of patients with dose modification will be summarized with the respective reasons for each treatment group. Frequency of dose modification will be summarized by categories.

Patient data listings will be provided for all dosing records and for calculated summary statistics.

### 7.4.2 Adverse Events

The adverse event verbatim descriptions (as recorded by the investigator on the eCRF) will be classified into standardized medical terminology using Medical Dictionary for Regulatory Activities (MedDRA). Adverse events will be coded to MedDRA by lower-level term, preferred term, and primary system organ class.

A treatment-emergent adverse event is defined as an adverse event that had an onset date or a worsening in severity from baseline (pretreatment) on or after the first dose of study drug and up to 30 days after study drug discontinuation. Only those adverse events that were treatment-emergent will be included in summary tables. All adverse events, treatment-emergent or otherwise, will be presented in patient data listings.

The incidence of treatment-emergent adverse events will be reported as the number (percentage) of patients with treatment-emergent adverse events by system organ class and preferred term. A patient will be counted only once by the highest severity within a system organ class and preferred term, even if the patient experienced more than 1 treatment-emergent adverse event within a specific system organ class and preferred term. The number (percentage) of patients with treatment-emergent adverse events will also be summarized by relationship to the study drug. Treatment-related treatment-emergent adverse events include those events considered by the investigator to be related to study drug or with missing assessment of the causal relationship. Serious adverse events, deaths, treatment-related treatment-emergent adverse events, and treatment-emergent adverse events that led to treatment discontinuation or dose modification will be summarized.

### 7.4.3 Laboratory Analyses

Clinical laboratory (chemistry, hematology, coagulation, or urinalysis) values will be evaluated for each laboratory parameter by treatment group as appropriate. Abnormal laboratory values will be flagged and identified as those outside (above or below) the normal range. Reference (normal) ranges for laboratory parameters will be provided. Descriptive summary statistics (eg, n, mean, standard deviation, median, minimum, maximum for continuous variables; n [%] for categorical variables) for laboratory parameters and their changes from baseline will be calculated. Laboratory values will be summarized by visit. Some parameters will also be plotted over time (eg, serum anti-PLA2R Ab titer, eGFR, serum albumin, and UPCR [based on 24-hour urine collection]). Laboratory parameters that are graded as mild, moderate, or severe will be summarized by severity. In the summary of laboratory parameters by severity, parameters with severity in both high and low directions will be summarized separately.

### 7.4.4 Vital Signs

Descriptive statistics for vital sign parameters (body temperature, heart rate, systolic and diastolic blood pressure, and weight) and changes from baseline will be presented by visit and treatment group. Vital signs will be listed by patient and visit.

### 7.5 Pharmacokinetic Analyses

PK samples will be collected to determine the concentrations of zanubrutinib at the timepoints specified in Table 9. The plasma concentrations at specific time windows and their statistics (eg, sample size, mean, standard deviation, coefficient of variance, median, minimum, maximum, and geometric mean) for each treatment group may be described in the Clinical Study Report.

The PK data may also be used for additional analyses such as population PK analyses and the results of such analyses may be reported separately from the Clinical Study Report.

Table 9 depicts the Schedule of Assessments specifying the different timepoints when the PK samples are collected as specified in peripheral blood samples and urine samples are collected from patients to explore the association of biomarkers with glomerular disease pathogenesis, disease activity, and the response to zanubrutinib.

TABLE 9
Sampling for pharmacokinetics and biomarkers

| | W 1 D 1 | | | W 4 | | | | | | | |
|---|---|---|---|---|---|---|---|---|---|---|---|
| | | | 4 to 6 | | | 4 to 6 | | | | | W 104/ |
| | Predose | 2 hours | hours | | 2 hours | hours | W 24 | W 52 | W 65 D 1 | W 76 | EOT |
| Assessments | (baseline) | postdose | postdose | Predose | postdose | postdose | Predose | Predose | Visit | Visit | Visit |
| PK sampling ^{a} | X ^{b} | X ^{c} | X ^{c} | X | X ^{c} | X ^{c} | | | | | |
| BTK | X | | | X | | | X | | X | | |
| occupancy ^{d, e} | | | | | | | | | | | |
| Serum | X | | | X | | | X | X | X | X | X |
| biomarker | | | | | | | | | | | |
| exploration | | | | | | | | | | | |
| (cytokines | | | | | | | | | | | |
| and | | | | | | | | | | | |
| proteomic | | | | | | | | | | | |
| signatures) ^{d} | | | | | | | | | | | |
| BTK-related | X | | | X | | | X | X | X | X | X |
| immune cell | | | | | | | | | | | |
| subpopulation ^{d} | | | | | | | | | | | |
| Blood gene | X | | | X | | | X | X | X | X | X |
| expression | | | | | | | | | | | |
| signature ^{d} | | | | | | | | | | | |
| Primary | X | | | | | | | | | | |
| membranous | | | | | | | | | | | |
| nephropathy- | | | | | | | | | | | |
| associated | | | | | | | | | | | |
| genetic | | | | | | | | | | | |
| polymorphism | | | | | | | | | | | |
| analysis ^{f} | | | | | | | | | | | |
| Urine | X | | | | | | X | X | X | X | X |
| biomarkers ^{d, g} | | | | | | | | | | | |

Abbreviations: BTK, Bruton tyrosine kinase; D, Day; EOT, end of treatment; PK, pharmacokinetics; W, Week.
^{a} Blood samples for PK analysis will be collected from all patients in Part 1 and randomized patients in Group A and Group B of Part 2 who received zanubrutinib treatment.
^{b} Within 30 minutes before administration of study drug.
^{c} A time window of ±30 minutes is allowed.
^{d} If W 1 D 1 predose (baseline) sample has not been collected, all the following samples will not be collected. For timepoints after W 1 D 1 predose (baseline), if sample collection is missed for certain reasons, unscheduled sample collection may be supplemented per sponsor's request during the following visit. Such unscheduled sample collection should not be regarded as protocol deviations.
^{e} Blood samples for BTK occupancy will be collected from patients in Part 1 and Part 2 (both Group A and Group B), with approximately 10 to 15 patients for each group and approximately 30-45 patients in total.
^{f} Blood samples will be collected for primary membranous nephropathy-associated genetic polymorphism analysis.
^{g} Patients should fast for ≥4 hours before urine sample collection. Avoid sample collection during menstruation.

7.6 Other Exploratory Analysis

Exploratory biomarker analyses may be performed to understand the association of these markers with study drug response.

### 7.7 Multiple Testing Strategy

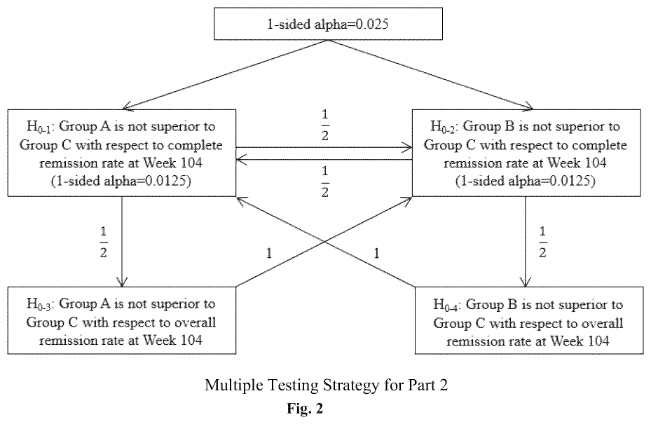

The graphical approach with a Bonferroni mixture of weighted Simes tests will be used for multiplicity adjustment in Part 2 as shown in FIG. 2. The 1-sided alpha of 0.0125 will be initially allocated to each primary null hypothesis. The testing of primary null hypotheses will be performed first. The secondary null hypothesis H₀₋₃ will only be tested if the corresponding primary null hypothesis H₀₋₁ is rejected. Similarly, H₀₋₄ will only be tested if H₀₋₂ is rejected. The arrows on the diagram show how the alpha allocated to a null hypothesis that is successfully rejected will be redistributed for the testing of other hypotheses. The weight for each test path indicates the fraction of the preserved alpha to be shifted along that path to the receiving hypothesis when the hypothesis at the tail end of the path is successfully rejected.

### 7.8 Sample Size Consideration

### Part 1:

Approximately 30 patients will be recruited into Part 1. With data from 30 patients for the analysis of reduction in UPCR at Week 24 from baseline, assuming the standard deviation is 2, if the mean reduction at Week 24 from baseline is less than 1.2, the posterior probability that the mean reduction is equal to or larger than 1.5 is less than 20%, and the study may be considered for stopping, along with the review of totality of the data from other efficacy and safety endpoints.

### Part 2:

The sample size calculation is based on the following assumptions:
1. Complete remission rate at Week 104: 5% for Group C and 22% for Group A or Group B
2. Overall remission rate at Week 104: 25% for Group C and 50% for Group A or Group B
3. 1-sided alpha of 0.025
4. Randomization ratio of 1:1:1

With these assumptions, a total of 252 randomized patients are needed to achieve approximately 90% power for the targeted treatment effect of the complete remission rate in assumption 1 and 82% power for the targeted treatment effect of the overall remission rate in assumption 2. Simulations were performed to determine the sample size using R software, version 3.6.1.

### Example 2

### 1. Research Purpose

Study the efficacy and mechanism of zanubrutinib in the treatment of primary membranous nephropathy by using related animal models.

### 2. Overall Design of the Study

In this study, two animal models of passive Heyman nephritis and transgenic mice are used to investigate whether zanubrutinib can alleviate the nephritis state of animals, and to explore the possible mechanism of zanubrutinib in the treatment of membranous nephropathy (MN). The experimental research plan includes: constructing a nephritis disease model, using zanubrutinib and control drugs to treat the disease model, and collecting and testing blood, urine, tissue and other samples in the disease model.

### 3. Animal Experiment Steps

### 3.1 Passive Hyman Nephritis Rat Model

3.1.1 Construction of passive Heymann nephritis (Heymann nephritis, HN) rat model

5-week-old male Sprague-Dawley rats (Viton Lever Laboratory Animal Technology Co., Ltd.), weighing about 130-180 g, are injected with sheep anti-Fx1A antibody serum 0.8 ml/100 g (Ptobetex, USA) in a single tail vein to construct MN mouse model.

### 3.1.2 Experimental Design of Intervention Therapy

48 rats are divided into 6 groups with 8 rats in each group. 5 groups of rats are used to construct nephritis disease models, and the 6th group is injected with the same dose of sterile normal saline as a negative control group. At the same time of inducing the disease in the rats, different treatments are given continuously from Day 0 by gavage until the rats are sacrificed on Day 42. The disease control group (group 1) is given normal saline; the low-dose group (group 2) of zanubrutinib is given twice a day (BID) 0.3 mg/kg/d, and the middle-dose group (group 3) is given daily Twice (BID) 3 mg/kg/d, the middle and high dose group (group 4) is given twice a day (BID) 30 mg/kg/d; the drug control group (group 5) is given once a day he Cromos. The zanubrutinib treatment group (groups 2-4) is administered twice a day with an interval of more than 8 hours. The disease control group (group 1) and the drug control group (Group 5) are injected with saline or tacrolimus once a day.

From the treatment groups (groups 2-4) on Day 0 and Day 7, before each administration of zanubrutinib (recorded as 0 hours), and 0.5, 1, 2, 4, and 8, 24 hours, after administration, a total of 7 time points, 80 ul of whole blood is taken from each rat in the three groups (24 rats in total) at each time point, and at least 30 ul plasma, marked with group numbers and time points, placed in cryovials, and stored at −80 oC. Zanubrutinib pharmacokinetics are measured by liquid chromatography-mass spectrometry.

### 3.1.3 Intervention Effect of Zanubrutinib

3.1.3.1 Collect urine and blood samples of rats every week, and record the body weight of rats. Detection of rat 24 h proteinuria, serum creatinine, blood urea nitrogen, albumin, cholesterol or triglyceride, liver enzymes (AST, ALT), cardiac enzymes (CK/CK-MB, LDH), specific anti-sheep IgG in circulation, Total IgG levels to assess disease severity in rats.
3.1.3.2 After the experiment, the fresh lymph nodes and spleen of the rats are taken, lymphocytes are extracted, and the T and B cell subsets are detected by flow cytometry to evaluate the autoimmune activation.
3.1.3.3 After the experiment, the kidney tissues of the rats are separated, and some kidney tissues are taken to detect the microstructure lesions of renal podocytes and basement membrane, and the staining of renal immunoglobulin and complement molecules by electron microscopy, immunofluorescence, immunohistochemistry and other methods to assess kidney damage.
3.1.3.4 According to the actual curative effect and the experimental results of the sections above, consider whether it is necessary to extract protein and RNA from some fresh kidney tissues for proteomics, RNA-seq detection of renal cortex cytokines, and pathway protein changes.

### 3.2 PLA2R Transgenic Mouse Model

### 3.2.1 Construction of PLA2R Transgenic MN Mouse Model

5-week-old PLA2R transgenic mice are injected with rabbit anti-human PLA2R antibody serum by a single tail vein injection to establish MN mouse model.

### 3.2.2 Experimental Design of Each Group

Set up 4 groups, of which the 1-3 groups have 6 animals each, and build the MN model. The disease control group (group 1) is given normal saline, the drug control group (group 2) is given tacrolimus by gavage from Day 0, and the zanubrutinib group (group 3) is given zanubrutinib by gavage Stomach Way from Day 0. Zanubrutinib (at the doses explored in the Hyman nephritis rat model) is administered continuously daily starting at Day 0. In the negative control group (group 4) 4 rats are injected with the same dose of sterile normal saline until they are sacrificed. 22 in total.

### 3.2.3 Intervention Effect of Zanubrutinib

3.2.3.1 Urine and blood samples from mice are collected weekly. Detect 24 h proteinuria, serum creatinine, blood urea nitrogen, albumin, cholesterol or triglyceride, liver enzymes, myocardial enzymes, etc., to assess the severity of the disease.
3.2.3.2 The mice are sacrificed to obtain kidney tissue, and the microstructure lesions of renal podocytes and basement membrane are detected by electron microscopy, immunofluorescence, and immunohistochemistry, and the staining of immunoglobulin and complement molecules is used to evaluate kidney damage.
3.2.3.3 Fresh lymph nodes and spleen are taken, lymphocytes are extracted, and flow cytometry of T and B cell subsets is performed to evaluate autoimmune activation.
3.2.3.4 Taking fresh spleen, from the aspects of zanubrutinib inhibition of B cell development and maturation, reduction of specific antibody production, inhibition of immune response, and reduction of complement level response activation, proteomics and RNA-seq experiments are used to explore the mechanisms for the treatment of MN with zanubrutinib.

### Example 3

The solid form of Compound A used herein is Form A of Compound 1 of WO2018/033853.

A number of references have been cited, the disclosures of which were incorporated herein by reference in their entirety.

What was claimed was:

1. A method of treating primary membranous nephropathy (pMN) comprising administering to a patient having primary membranous nephropathy a therapeutically effective amount of a Bruton's Tyrosine Kinase (BTK) inhibitor.

2. The method of claim 1, wherein the Bruton's Tyrosine Kinase inhibitor is Compound A having the name of (S)-7-(1-acryloylpiperidin-4-yl)-2-(4-phenoxyphenyl)-4,5,6,7-tetrahydropyrazolo[1,5-a]pyrimidine-3-carboxamide or the following structure: 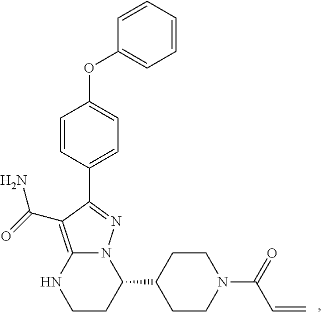 [SMILES: C=CC(=O)N1CCC([C@@H]2CCNc3c(C(N)=O)c(-c4ccc(Oc5ccccc5)cc4)nn32)CC1]
or a pharmaceutically acceptable salt, tautomer, stereoisomer, enantiomer, isotopologue, solvate, or prodrug thereof.

3. The method of claim 2, wherein the patient is identified as having (i) an elevated level of an autoantibody specific for an antigen or (ii) kidney tissue comprising an elevated level of the antigen, wherein the antigen is PLA2R, or THSD7A.

4. The method of any one of claims 1-3, wherein the patient is identified as biopsy confirmed primary membranous nephropathy and with persistent nephrotic syndrome after run-in period.

5. The method of any one of claims 1-4, wherein the patient is identified as having an elevated level of the autoantibody.

6. The method of any one of claims 1-5, wherein the level of autoantibodies present within the patient is reduced by at least about 5 percent, at least about 25 percent, or at least about 50 percent following the administering step.

7. The method of any one of claims 3-6, wherein the antigen is PLA2R.

8. The method of claim 7, wherein the patient is a PLA2R mediated patient with primary membranous nephropathy.

9. The method of claim 7, wherein the level of an autoantibody specific for PLA2R is more than about 20 RU/mL, about 40 RU/mL, about 50 RU/mL, about 60 RU/mL, or about 80 RU/mL in serum prior to the administering step.

10. The method of any one of claims 3-6, wherein the antigen is THSD7A.

11. The method of claim 10, wherein the patient is a THSD7A mediated patient with primary membranous nephropathy.

12. The method of any one of claims 1-3, wherein the patient is identified as having the kidney tissue comprising an elevated level of the antigen.

13. The method of claim 12, wherein the level of the antigen present within kidney tissue is reduced by at least about 5 percent, at least about 25 percent, or at least about 50 percent following the administering step.

14. The method of any one of claims 1-3 and 11-13, wherein the antigen is PLA2R.

15. The method of any one of claims 1-3 and 11-13, wherein the antigen is THSD7A.

16. The method of any one of claims 2-15, wherein Compound A or a pharmaceutically acceptable salt, tautomer, stereoisomer, enantiomer, isotopologue, solvate, or prodrug thereof is administered one, two, or three times a day.

17. The method any one of claims 2-16, wherein Compound A or a pharmaceutically acceptable salt, tautomer, stereoisomer, enantiomer, isotopologue, solvate, or prodrug thereof is administered from about 40 mg to about 320 mg per day.

18. The method of claim 17, wherein Compound A or a pharmaceutically acceptable salt, tautomer, stereoisomer, enantiomer, isotopologue, solvate, or prodrug thereof is administered at about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, or about 320 mg per day.

19. The method of claim 18, wherein Compound A or a pharmaceutically acceptable salt, tautomer, stereoisomer, enantiomer, isotopologue, solvate, or prodrug thereof is administered at about 40 mg, about 80 mg, about 160 mg, or about 320 mg per day.

20. The method of claim 19, wherein the method comprises administering to the patient Compound A at a dose of about 160 mg twice a day (BID) or about 160 mg once a day (QD).

21. The method of claim 19, wherein the method comprises administering to the patient Compound A at a dose of about 160 mg twice a day (BID) or about 160 mg once a day (QD) orally.

22. The method of claim 19, wherein the method comprises administering to the patient Compound A at a dose of about 160 mg twice a day (BID) orally.

23. The method of claim 19, wherein the method comprises administering to the patient Compound A at a dose of about 160 mg once a day (QD) orally.

24. The method of any one of claims 1-20, wherein the patient achieves a partial remission, or a complete remission.

25. The method of claim 24, wherein the patient achieves a partial remission.

26. The method of claim 24, wherein the patient achieves a complete remission.

27. The method of any one of claims 1-20, wherein the method provides a plasma Compound A AUC₀₋₂₄ₕ between about 1,607 ng*h/ml and about 2,984 ng*h/ml in the patient.

28. The method of any one of claims 1-20, wherein the method provides a plasma Compound A AUC_{0-∞}, between about 843 ng*h/ml and about 3,786 ng*h/ml in the patient.
